SEQUENCE LISTING (43 sequences)

SEQ ID NO: 1 - protein, 30 aa, Saccharomyces cerevisiae
PEDTVDFLKEVESRMQHITQGRTSMKIQFK

SEQ ID NO: 2 - protein, 28 aa, Saccharomyces cerevisiae
PPTEVLLESIQSKVESIEQLQRKLQHVQ

SEQ ID NO: 3 - protein, 22 aa, Saccharomyces cerevisiae
EQQNNEMCSTLCHHSLPALIEG

SEQ ID NO: 4 - protein, 25 aa, Saccharomyces cerevisiae
HHSLPALIEGQRKYYADYYAYRQEI

SEQ ID NO: 5 - protein, 12 aa, Homo sapiens
EKKMKILLGGYQ

SEQ ID NO: 6 - protein, 12 aa, Drosophila melanogaster
EKKLKILTGGYZ

SEQ ID NO: 7 - protein, 12 aa, Lentinula (Fungi)
EKKLGKVLGGYD

SEQ ID NO: 8 - protein, 12 aa, Plasmodium sp.
ENKYDIYTKGYQ

SEQ ID NO: 9 - protein, 30 aa, Homo sapiens
PYLFSCCEDKQVKCWDLEYNKVIRHYHGHL

SEQ ID NO: 10 - protein, 30 aa, Homo sapiens
PQIITGSHDTTIRLWDLVAGKTRVTLTNHK

SEQ ID NO: 11 - protein, 24 aa, Homo sapiens
HMTTEAKRAAKMEKKMKILLGGYQ

SEQ ID NO: 12 - protein, 24 aa, Homo sapiens
ELKKHEDSAIPRRLECLKEDVQRQ

SEQ ID NO: 13 - protein, 24 aa, Homo sapiens
EREKELQHRYADLLLEKETLKSKF

SEQ ID NO: 14 - protein, 24 aa, Homo sapiens
QYGGLLIKMKKEMKAARKAETTMH

SEQ ID NO: 15 - protein, 12 aa, Homo sapiens
HMTTEAKRAAKM

SEQ ID NO: 16 - protein, 12 aa, Homo sapiens
KRAAKMEKKMKI

SEQ ID NO: 17 - protein, 12 aa, Homo sapiens
QYGGLLIKMKKE

SEQ ID NO: 18 - protein, 8 aa, Homo sapiens
KILLGGYQ

SEQ ID NO: 19 - protein, 12 aa, Drosophila melanogaster
EKKLKILTGGYQ

SEQ ID NO: 20 - protein, 12 aa, Lentinula (Fungi)
EKKLGKVLGGYQ

SEQ ID NO: 21 - protein, 590 aa, Saccharomyces cerevisiae
MPPVPIYVKGGVWTNVEDQILKAAVQKYGTHQWSKVASLLQKKTARQSELRWNEYLNPKLNFTEFSKEEDAQLLDLARELPNQWRTIADMMARPAQVCVERYNRLLESEDSGGAALSTGVTDLKAGDINPNAETQMARPDNGDLEDEEKEMLAEARARLLNTQGKKATRKIRERMLEESKRIAELQKRRELKQAGINVAIKKPKKKYGTDIDYNEDIVYEQAPMPGIYDTSTEDRQIKKKFEQFERKVNRKGLDGNKDKPSKKNKDKKRKHDENEHVEKAALGESTTLTDEYKKPKLILSAPGTKQGKVTYKKKLESKRQKLIEAQATGTVLTPKELLPHDSGQEDNERSNIKSGKQLKSRIRKFLVQMFASLPSPKNDFEIVLSEDEKEEDAEIAEYEKEFENERAMNEEDNFIEPPSQNDAPRVSLVAVPLAYSTLPIPEFKNNPQSAIDNKYNLLVANAINKEPHMVPEDTVDFLKEVESRMQHITQGRTSMKIQFKTAMPPTEVLLESIQSKVESIEQLQRKLQHVQPLEQQNNEMCSTLCHHSLPALIEGQRKYYADYYAYRQEIRSLEGRRKRLQAMLNSSSSI

SEQ ID NO: 22 - protein, 757 aa, Schizosaccharomyces pombe
MVVLKGGAWKNTEDEILKAAVSKYGKNQWARISSLLVRKTPKQCKARWYEWIDPSIKKTEWSREEDEKLLHLAKLLPTQWRTIAPIVGRTATQCLERYQKLLDDLEAKENEQLGLISGEGAEAAAPVNDPNSRLRFGEAEPNLETLPALPDAIDMDEDEKEMLSEARARLANTQGKKAKRKDREKQLELTRRLSHLQKRRELKAAGINIKLFRRKKNEMDYNASIPFEKKPAIGFYDTSEEDRQNFREKREADQKIIENGIRNNEMESEGRKFGHFEKPKPIDRVKKPNKDAQEEKMRRLAEAEQMSKRRKLNLPSPTVSQDELDKVVKLGFAGDRARAMTDTTPDANYSTNLLGKYTQIERATPLRTPISGELEGREDSVTIEVRNQLMRNREQSSLLGQESIPLQPGGTGYTGVTPSHAANGSALAAPQATPFRTPRDTFSINAAAERAGRLASERENKIRLKALRELLAKLPKPKNDYELMEPRFADETDVEATVGVLEEDATDRERRIQERIAEKERLAKARRSQVIQRDLIRPSVTQPEKWKRSLENEDPTANVLLKEMIALISSDAINYPFGNSKVKGTANKVPDLSNEEIERCRLLLKKEIGQLESDDYIQFEKEFLETYSALHNTSSLLPGLVIYEEDDEDVEAAEKFYTNDIQRDLAKKALECNKLENRVYDLVRSSYEQRNFLIKKISHAWKALQTERKNLTCYEFLYNQERLALPNRLEAAEIELSKMQQIEAYAQQDYARVTGQN

SEQ ID NO: 23 - protein, 844 aa, Arabidopsis thaliana
MRIMIKGGVWKNTEDEILKAAVMKYGKNQWARISSLLVRKSAKQCKARWYEWLDPSIKKTEWTREEDEKLLHLAKLLPTQWRTIAPIVGRTPSQCLERYEKLLDAACTKDENYDAADDPRKLRPGEIDPNPEAKPARPDPVDMDEDEKEMLSEARARLANTRGKKAKRKAREKQLEEARRLASLQKRRELKAAGIDGRHRKRKRKGIDYNAEIPFEKRAPAGFYDTADEDRPADQVKFPTTIEELEGKRRADVEAHLRKQDVARNKIAQRQDAPAAILQANKLNDPEVVRKRSKLMLPPPQISDHELEEIAKMGYASDLLAENEELTEGSAATRALLANYSQTPRQGMTPMRTPQRTPAGKGDAIMMEAENLARLRDSQTPLLGGENPELHPSDFTGVTPRKKEIQTPNPMLTPSMTPGGAGLTPRIGLTPSRDGSSFSMTPKGTPFRDELHINEDMDMQQSAKLERQRREEARRSLRSGLTGLPQPKNEYQIVAQPPPEESEEPEEKIEEDMSDRIAREKAEEEARQQALLKKRSKVLQRDLPRPPAASLAVIRNSLLSADGDKSSVVPPTPIEVADKMVREELLQLLEHDNAKYPLDDKAEKKKGAKNRTNRSASQVLAIDDFDENELQEADKMIKEEGKFLCVSMGHENKTLDDFVEAHNTCVNDLMYFPTRSAYELSSVAGNADKVAAFQEEMENVRKKMEEDEKKAEHMKAKYKTYTKGHERRAETVWTQIEATLKQAEIGGTEVECFKALKRQEEMAASFRKKNLQEEVIKQKETESKLQTRYGNMLAMVEKAEEIMVGFRAQALKKQEDVEDSHKLKEAKLATGEEEDIAIAMEASA

SEQ ID NO: 24 - protein, 755 aa, Caenorhabditis elegans
MVRVIIKGGVWKNTEDEILKAAIMKYGKNQWSRIASLLHRKSAKQCKARWFEWLDPGIKKTEWSREEDEKLLHLAKLMPTQWRTIAPIVGRTSAQCLERYEHLLDEAQRKAEGLDEEATETRKLKPGEIDPTPETKPARPDPIDMDDDELEMLSEARARLANTQGKKAKRKARERQLSDARRLASLQKRREMRAAGLAFARKFKPKRNQIDYSEEIPFEKHVPAGFHNPSEDRYVVEDANQKAIEDHQKPRGREIEMEMRREDREKLKKRKEQGEADAVFNIKEKKRSKLVLPEPQISDRELEQIVKIGHASDSVRQYIDGTATSGLLTDYTESARANAVAARTMRTPMLKDTVQLELENLMALQNTESALKGGLNTPLHESELGKGVLPTPKVAATPNTVLHAIAATPGTQSQFPGSTPGGFATPAGSVAATPFRDQMRINEEIAGSALEQKASLKRALASLPTPKNDFEVVGPDDDEVEGAVEDESNQDEDGWIEDASERAENKAKRNAENRVRNMKMRSQVIQRSLPKPTKVNEQATRATNSSADDMVKAEMSKLLAWDVDNKPPSVIYSREELDAAADLIKQEAESGPELNSLMWKVVEQCTSEIILSKDKFTRIAILPREEQMKALNDEFQMYRGWMNQRAKRAAKVEKKLRVKLGGYQAIHDKLCKKYQEVTTEIEMANIEKKTFERLGEHELKAINKRVGRLQQEVTTQETREKDLQKMYSKLSNKQWKLSQIEIHDAASTTSAPITY

SEQ ID NO: 25 - protein, 791 aa, Drosophila melanogaster
MKYGKNQWSRIASLLHRKSAKQCKARWYEWLDPSIKKTEWSREEDEKLLHLAKLMPTQWRTIAPIIGRTAAQCLERYEYLLDQAQRKEDGEDTMDDPRKLKPGEIDPNPETKPARPDPKDMDEDELEMLSEARARLANTQGKKAKRKAREKQLEEARRLATLQKRRELRAAGIGSGNRKRIKGIDYNAEIPFEKRPAHGFYDTSEEHLQKNEPDFNKMRQQDLDGELRSEKEERERKRDKQKLKQRKENEVPTAMLQNMEPERKRSKLVLPTPQISDMELQQVVKLGRASEMAKEIAGESGIETTDALLADYSITPQVAATPRTPAPYTDRIMQEAQNMMALTHTETPLKGGLNTPLHESDFSGVLPKAASIATPNTVIATPFRTQREGGAATPGGFQTPSSGALVPVKGAGGATGVVNTPAYVRDKLSINPEESMGVTETPAHYKNYQKQLKSTLRDGLSTLPAPRNDYEIVVPEQEESERIETNSEPAVEDQADVDARLLAEQEARRKRELEKRSQVIQRSLPRPTEVNTKILRPQSEKQNLTEQQQAEELIKHEMITMQLYDSVKDPVPGQSQHKLEQLQSYFKANPYEDISQQELAKAKQMLTEEMEVVKERMAHGELPLDVYAQVWQECLGQVLYLPSQHRYTRANLASKKDRLESAEKRLETNRRHMAKEAKRCGKIEKKLKILTGGYQARAQVLIKQLQDTYGQIEQNSVSLSTFRFLGEQEAIAVPRRLESLQEDVRRQMDREKELQQKYASLVEERDSLYSQIEHITGVRPTAQQLLPDQEA

SEQ ID NO: 26 - protein, 802 aa, Homo sapiens
MPRIMIKGGVWRNTEDEILKAAVMKYGKNQWSRIASLLHRKSAKQCKARWYEWLDPSIKKTEWSREEEEKLLHLAKLMPTQWRTIAPIIGRTAAQCLEHYEFLLDKAAQRDNEEETTDDPRKLKPGEIDPNPETKPARPDPIDMDEDELEMLSEARARLANTQGKKAKRKAREKQLEEARRLAALQKRRELRAAGIEIQKKRKRKRGVDYNAEIPFEKKPALGFYDTSEENYQALDADFRKLRQQDLDGELRSEKEGRDRKKDKQHLKRKKESDLPSAILQTSGVSEFTKKRSKLVLPAPQISDAELQEVVKVGQASEIARQTAEESGITNSASSTLLSEYNVTNNSVALRTPRTPASQDRILQEAQNLMALTNVDTPLKGGLNTPLHESDFSGVTPQRQVVQTPNTVLSTPFRTPSNGAEGLTPRSGTTPKPVINSTPGRTPLRDKLNINPEDGMADYSDPSYVKQMERESREHLRLGLLGLPAPKNDFEIVLPENAEKELEEREIDDTYIEDAADVDARKQAIRDAERVKEMKRMHKAVQKDLPRPSEVNETILRPLNVEPPLTDLQKSEELIKKEMITMLHYDLLHHPYEPSGNKKGKTVGFGTNNSEHITYLEHNPYEKFSKEELKKAQDVLVQEMEVVKQGMSHGELSSEAYNQVWEECYSQVLYLPGQSRYTRANLASKKDRIESLEKRLEINRGHMTTEAKRAAKMEKKMKILLGGYQSRAMGLMKQLNDLWDQIEQAHLELRTFEELKKHEDSAIPRRLECLKEDVQRQQEREKELQHRYADLLLEKETLKSKF

SEQ ID NO: 27 - protein, 150 aa, Saccharomyces cerevisiae
PEFKNNPQSAIDNKYNLLVANAINKEPHMVPEDTVDFLKEVESRMQHITQGRTSMKIQFKTAMPPTEVLLESIQSKVESIEQLQRKLQHVQPLEQQNNEMCSTLCHHSLPALIEGQRKYYADYYAYRQEIRSLEGRRKRLQAMLNSSSSI

SEQ ID NO: 28 - protein, 152 aa, Schizosaccharomyces pombe
KEIGQLESDDYIQFEKEFLETYSALHNTSSLLPGLVIYEEDDEDVEAAEKFYTNDIQRDLAKKALECNKLENRVYDLVRSSYEQRNFLIKKISHAWKALQTERKNLTCYEFLYNQERLALPNRLEAAEIELSKMQQIEAYAQQDYARVTGQN

SEQ ID NO: 29 - protein, 154 aa, Arabidopsis thaliana
AAFQEEMENVRKKMEEDEKKAEHMKAKYKTYTKGHERRAETVWTQIEATLKQAEIGGTEVECFKALKRQEEMAASFRKKNLQEEVIKQKETESKLQTRYGNMLAMVEKAEEIMVGFRAQALKKQEDVEDSHKLKEAKLATGEEEDIAIAMEASA

SEQ ID NO: 30 - protein, 151 aa, Caenorhabditis elegans
CTSEIILSKDKFTRIAILPREEQMKALNDEFQMYRGWMNQRAKRAAKVEKKLRVKLGGYQAIHDKLCKKYQEVTTEIEMANIEKKTFERLGEHELKAINKRVGRLQQEVTTQETREKDLQKMYSKLSNKQWKLSQIEIHDAASTTSAPITY

SEQ ID NO: 31 - protein, 152 aa, Drosophila melanogaster
YLPSQHRYTRANLASKKDRLESAEKRLETNRRHMAKEAKRCGKIEKKLKILTGGYQARAQVLIKQLQDTYGQIEQNSVSLSTFRFLGEQEAIAVPRRLESLQEDVRRQMDREKELQQKYASLVEERDSLYSQIEHITGVRPTAQQLLPDQEA

SEQ ID NO: 32 - protein, 151 aa, Homo sapiens
LSSEAYNQVWEECYSQVLYLPGQSRYTRANLASKKDRIESLEKRLEINRGHMTTEAKRAAKMEKKMKILLGGYQSRAMGLMKQLNDLWDQIEQAHLELRTFEELKKHEDSAIPRRLECLKEDVQRQQEREKELQHRYADLLLEKETLKSKF

SEQ ID NO: 33 - protein, 451 aa, Saccharomyces cerevisiae
MDGNDHKVENLGDVDKFYSRIRWNNQFSYMATLPPHLQSEMEGQKSLLMRYDTYRKESSSFSGEGKKVTLQHVPTDFSEASQAVISKKDHDTHASAFVNKIFQPEVAEELIVNRYEKLLSQRPEWHAPWKLSRVINGHLGWVRCVAIDPVDNEWFITGSNDTTMKVWDLATGKLKTTLAGHVMTVRDVAVSDRHPYLFSVSEDKTVKCWDLEKNQIIRDYYGHLSGVRTVSIHPTLDLIATAGRDSVIKLWDMRTRIPVITLVGHKGPINQVQCTPVDPQVVSSSTDATVRLWDVVAGKTMKVLTHHKRSVRATALHPKEFSVASACTDDIRSWGLAEGSLLTNFESEKTGIINTLSINQDDVLFAGGDNGVLSFYDYKSGHKYQSLATREMVGSLEGERSVLCSTFDKTGLRLITGEADKSIKIWKQDETATKESEPGLAWNPNLSAKRF

SEQ ID NO: 34 - protein, 486 aa, Arabidopsis thaliana
MPAPTTEIEPIEAQSLKKLSLKSLKRSLELFSPVHGQFPPPDPEAKQIRLSHKMKVAFGGVEPVVSQPPRQPDRINEQPGPSNALSLAAPEGSKSTQKGATESAIVVGPTLLRPILPKGLNYTGSSGKSTTIIPANVSSYQRNLSTAALMERIPSRWPRPEWHAPWKNYRVIQGHLGWVRSVAFDPSNEWFCTGSADRTIKIWDVATGVLKLTLTGHIEQVRGLAVSNRHTYMFSAGDDKQVKCWDLEQNKVIRSYHGHLSGVYCLALHPTLDVLLTGGRDSVCRVWDIRTKMQIFALSGHDNTVCSVFTRPTDPQVVTGSHDTTIKFWDLRYGKTMSTLTHHKKSVRAMTLHPKENAFASASADNTKKFSLPKGEFCHNMLSQQKTIINAMAVNEDGVMVTGGDNGSIWFWDWKSGHSFQQSETIVQPGSLESEAGIYAACYDNTGSRLVTCEADKTIKMWKEDENATPETHPINFKPPKEIRRF

SEQ ID NO: 35 - protein, 494 aa, Caenorhabditis elegans
MSASVSDPYEQMPAAPTDDDLEDKPEADKKALLNQVFKSLKRAQDLFYHDYAQPPPMPEENDSLIRSMKRKHEYGNVIKKVEEMKVRRENEMLALPTSQPMHGTGSVIASAGTPLAITDGSGKLVNQQQGSAKSGTLLPLVPLGNSSKGEDNTTRSLLPSKAPMMMKPKWHAPWKLYRVASGHTGWVRAVDVEPGNQWFASGGADRIIKIWDLASGQLKLSLTGHISSVRAVKVSPRHPFLFSGGEDKQVKCWDLEYNKVIRHYHGHLSAVQALSVHPSLDVLVTCARDSTARVWDMRTKAQVHCFAGHTNTVADVVCQSVDPQVITASHDATVRLWDLAAGRSMCTLTHHKKSVRALTIHPRLNMFASASPDNIKQWKLPKGEFMQNLSGHNAIINTLSSNDDGVVVSGADNGSLCFWDWRSGFCFQKIQTKPQPGSIESEAGIYASCFDKTGLRLITAEADKTIKMYKEDDEATEESHPIVWRPEIVKKKAY

SEQ ID NO: 36 - protein, 514 aa, Homo sapiens
MVEEVQKHSVHTLVFRSLKRTHDMFVADNGKPVPLDEESHKRKMAIKLRNEYGPVLHMPTSKENLKEKGPQNATDSYVHKQYPANQGQEVEYFVAGTHPYPPGPGVALTADTKIQRMPSESAAQSLAVALPLQTKADANRTAPSGSEYRHPGASDRPQPTAMNSIVMETGNTKNSALMAKKAPTMPKPQWHPPWKLYRVISGHLGWVRCIAVEPGNQWFVTGSADRTIKIWDLASGKLKLSLTGHISTVRGVIVSTRSPYLFSCGEDKQVKCWDLEYNKVIRHYHGHLSAVYGLDLHPTIDVLVTCSRDSTARIWDVRTKASVHTLSGHTNAVATVRCQAAEPQIITGSHDTTIRLWDLVAGKTRVTLTNHKKSVRAVVLHPRHYTFASGSPDNIKQWKFPDGSFIQNLSGHNAIINTLTVNSDGVLVSGADNGTMHLWDWRTGYNFQRVHAAVQPGSLDSESGIFACAFDQSESRLLTAEADKTIKVYREDDTATEETHPVSWKPEIIKRKRF

SEQ ID NO: 37 - DNA, 2409 bp, Homo sapiens
atgcctcgaattatgatcaaggggggcgtatggaggaataccgaggatgaaattctgaaagcagcggtaatgaaatatgggaaaaatcagtggtctaggattgcctcattgctgcatagaaaatcagcaaagcagtgcaaagccagatggtatgaatggctggatccaagcattaagaagacagaatggtccagagaagaagaggaaaaactcttgcacttggccaagttgatgccaactcagtggaggaccattgctccaatcattggaagaacagcggcccagtgcttagaacactatgaatttcttctggataaagctgcccaaagagacaatgaagaggaaacaacagatgatccacgaaaacttaaacctggagaaatagatccaaatccagaaacaaaaccagcgcggcctgatccaattgatatggatgaggatgaacttgagatgctttctgaagccagagcccgcttggctaatactcagggaaagaaggccaagaggaaagcaagagagaaacaattggaagaagcaagacgtcttgctgccctccaaaaaagaagagaacttcgagcagctggcatagaaattcagaagaaaagaaaaaggaagagaggagttgattataatgccgaaatcccatttgaaaaaaagcctgcccttggtttttatgatacttctgaggaaaactaccaagctcttgacgcagatttcaggaaattaagacaacaggatcttgatggggagctaagatctgaaaaagaaggaagagatagaaaaaaagacaaacagcatttgaaaaggaaaaaagaatctgatttaccatcagctattcttcaaactagtggtgtttctgaatttactaaaaagagaagcaaactagtacttcctgcccctcagatttcagatgcagaactccaggaagttgtaaaagtaggccaagcgagtgaaattgcacgtcaaactgccgaggaatctggcataacaaattctgcttccagtacacttttgtctgagtacaatgtcaccaacaacagcgttgctcttagaacaccacgaacaccagcttcccaggacagaattctgcaggaagcccagaacctcatggccctcaccaatgtggacaccccattgaaaggtggacttaataccccattgcatgagagtgacttctcaggtgtaactccacagcgacaagttgtacagactccaaacacagttctctctactccattcaggactccttctaatggagctgaagggctgactccccggagtggaacaactcccaaaccagttattaactctactccgggtagaactcctcttcgagacaagttaaacattaatcccgaggatggaatggcagactatagtgatccctcttacgtgaagcagatggaaagagaatcccgagaacatctccgtttagggttgttgggccttcctgcccctaagaatgattttgaaattgttctaccagaaaatgccgagaaggagctggaagaacgtgaaatagatgatacttacattgaagatgctgctgatgtggatgctcgaaagcaggccatacgagatgcagagcgtgtaaaggaaatgaaacgaatgcataaagctgtccagaaagatctgccaagaccatcagaagtaaatgaaactattctaagacccttaaatgtagaaccgcctttaacagatttacagaaaagtgaagaactaatcaaaaaagaaatgatcacaatgcttcattatgaccttctacatcacccttatgaaccatctggaaataaaaaaggcaaaactgtagggtttggtaccaataattcagagcacattacctatctggaacataatccttatgaaaagttctccaaagaagagctgaaaaaggcccaggatgttttggtgcaggagatggaagtggttaaacaaggaatgagccatggagagctctcaagtgaagcttataaccaggtgtgggaagaatgctacagtcaagttttatatcttcctgggcagagccgctacacacgggccaatctggctagtaaaaaggacagaattgaatcacttgaaaagaggctcgagataaacaggggtcacatgacgacagaagccaagagggctgcaaagatggaaaagaagatgaaaattttgcttgggggttaccagtctcgtgctatggggctcatgaaacagttgaatgacttatgggaccaaattgaacaggctcacttggagttacgcacttttgaagaactcaagaaacatgaagattctgctattccccggaggctagagtgtctaaaagaagacgttcagcgacaacaagaaagagaaaaggaacttcaacatagatatgctgatttgctgctggagaaagagactttaaagtcaaaattctga

SEQ ID NO: 38 - DNA, 1773 bp, Saccharomyces cerevisiae
atgccccccgtaccaatatacgtgaaaggcggtgtatggaccaatgtggaggatcagattcttaaagcggctgtacaaaaatacggaactcatcagtggagcaaagtagcatcccttttgcagaaaaagactgccaggcagagtgaattaaggtggaatgaatatttaaatccaaagttgaattttacagagttctcgaaggaggaggatgcccaacttctagatcttgcaagagagctgcctaatcagtggaggaccatagctgatatgatggccaggcctgcacaggtctgcgtcgaaagatataataggctattagaaagtgaagacagtggaggggctgcgttaagtacaggagttactgacttgaaagccggggatattaatcctaacgctgaaactcaaatggctagaccagataatggtgatttggaagatgaggaaaaggaaatgcttgctgaagctagggctcgtctgttaaatacccaaggtaagaaggctacaagaaagattagagagcggatgctcgaagaatcaaaacgcattgctgaactacaaaagaggcgtgagctaaagcaagcaggaattaatgttgccattaagaaaccgaaaaaaaaatacggcaccgacattgattacaatgaagatatagtatatgaacaagctcccatgccaggtatatatgacacgtccactgaagaccgccagataaaaaagaagtttgagcagtttgagagaaaagtcaacagaaaaggtttggatggtaataaggataagccaagtaaaaaaaataaggacaaaaaaagaaaacacgatgagaacgaacacgttgagaaagcagcactgggtgaatctactacattgacggatgagtataaaaaaccaaaacttatactatctgcaccaggaacgaaacagggaaaagtcacctataaaaaaaagctagaaagcaaaagacaaaaactaatcgaggcacaagcaactggcactgtgttgacaccaaaagaactactaccccacgactccggccaagaagataatgaacgcagtaatataaagagtggtaaacagctaaaatcacgcatacgaaaatttttagtgcaaatgttcgcatctttgcctagtcccaagaacgatttcgaaattgtattaagtgaagacgagaaagaagaagatgcagagatagcggaatacgagaaagaatttgaaaatgaaagagcgatgaatgaagaggacaacttcatagaaccaccatctcaaaatgatgcgccacgcgtctcattagtagccgttccattagcttactcaacgctacccataccagaattcaaaaacaatccgcagtccgcgattgacaataagtacaacttgctagtcgcgaacgccataaacaaagaacctcacatggtaccagaagatacggtagattttctcaaagaggtggagtcgcgtatgcagcatataacccaagggcgcacttccatgaaaatacaattcaaaacagcaatgcccccaactgaggttcttctggaatcgatccaatcaaaagtggagtctattgaacagttacagcgtaaactacaacatgtgcaaccactggaacaacagaataacgagatgtgcagtaccctctgccatcacagcctgcccgctttgattgaagggcaacgcaagtactacgctgattactacgcctaccgacaagagatacgatcgcttgagggtcgtagaaagcgtcttcaagccatgctaaattcttcttcttccatatag

SEQ ID NO: 39 - DNA, 1740 bp, Schizosaccharomyces pombe
gctttctgacaattgtcagggttttggcgaatatttagaaaatggttgttttaaaaggaggtgcctggaaaaatacagaggatgaaatcttaaaggctgctgtcagtaaatacggtaaaaaccaatgggcccgcataagttcattgctggttcgcaaaactcctaaacaatgtaaagctcgttggtatgaatggatcgatccaagcattaaaaagactgaatggagtcgtgaagaggacgaaaaattattacacttagctaagttacttcctacacagtggcggacgattgctccaattgtaggtcggacggccactcaatgtttagaacgctatcaaaaacttcttgatgatttggaagcgaaagagaacgagcagttgggattgatcagtggagaaggtgcagaagcagctgcgcctgttaacgatccaaattcccggcttcgttttggcgaagcagagcccaatttagaaaccctacctgcactccctgatgccattgacatggatgaagacgaaaaagagatgttgagtgaagcacgtgcacgtttagcaaacacacaaggaaagaaagccaaacgaaaggatagagaaaagcagcttgaattaactagaagattgtcacatctccaaaagcgaagagaattgaaggcagctggaatcaatataaagctcttccgtcgaaagaagaatgaaatggattataacgcttctattccatttgaaaaaaagccagcgattggtttttatgatacctcagaagaggacaggcaaaattttcgggaaaagcgagaagcggaccagaagataattgaaaatggaatacggaataatgaaatggaatccgaaggtcgaaaatttggtcattttgaaaagccaaaacctattgatagagtaaaaaagcccaacaaagatgcccaagaagaaaaaatgcgacgtcttgccgaggctgagcagatgagcaagaggcggaagttgaatttacctagtcctactgtgtctcaagatgaactggataaagtagtgaaactaggctttgcaggcgatcgtgctcgcgctatgactgatacgactccagatgctaattacagtactaatttgttaggaaagtatacacaaattgaacgagctactccattgagaacaccaattagtggtgaacttgagggaagagaggattcagttacaattgaagttagaaatcaattgatgaggaaccgcgaacaatcttctctcttgggtcaggaaagtatacctttacaacctggtggtactggttacacaggagttactcccagtcatgcagctaatggctcagctttggcagcacctcaagcaactccttttagaacaccacgtgacacattttctattaatgcagctgcggagcgagcaggacgattagcaagtgaaagagaaaataaaattcgattgaaggctttgcgagaattgcttgcaaaactacctaaacctaaaaacgactatgaactaatggagcctcgatttgcggacgaaactgatgttgaagctactgttggagtgcttgaagaagacgcaacagaccgagaacgtcgtatccaagaacgtattgcagaaaaagaaaggttggcaaaagcgaggaggtcacaggttattcaaagagatttgattcgaccatctgtaacacaaccagaaaaatggaagcgttcacttgaaaatgaagatccgactgcaaatgttttattgaaagaaatgattgctt

SEQ ID NO: 40 - DNA, 1548 bp, Homo sapiens
atggtcgaggaggtacagaaacattctgtacacacccttgtgttcaggtcgttgaagaggacccatgacatgtttgtagctgataatggaaaacctgtgcctttagatgaagagagtcacaaacgaaaaatggcaatcaagcttcgtaatgagtatggtcctgtgttgcatatgcctacttcaaaagaaaatcttaaagagaagggtcctcagaatgcaacggattcatatgttcataaacagtaccctgccaatcaaggacaagaagttgaatactttgtggcaggtacacatccatacccaccaggacctggggttgctttgacagcagatactaagatccagagaatgccaagtgaatcagctgcacagtccttagcggtggcattacctttgcagaccaaggctgatgcaaatcgtactgcccctagtggaagtgaataccgacatcctggggcttctgaccgtccacagcctacagcgatgaattcaattgtcatggagactggcaataccaagaactctgcactgatggctaaaaaagcccctacaatgccaaaaccccagtggcacccaccgtggaaactctacagggttatcagtgggcatcttggctgggttcgatgtattgctgtggaacctggaaatcagtggtttgttactggatctgctgacagaactataaagatctgggacttggctagtggcaaattaaaactgtcattgactgggcatattagtactgtgcggggcgtgatagtaagcacaaggagcccatatctgttctcttgtggagaagacaaacaagtgaaatgctgggatctcgaatacaataaggttatacggcattatcatggacatttaagtgcagtgtatggtttggatttgcacccgacaatcgatgtgttggtaacctgtagtcgagattcaactgcacggatttgggatgtgagaactaaagccagtgtacacacattatctggacatacaaatgcagttgctacagtgagatgtcaggctgcagaaccacaaattattacaggaagccatgatactacaattcgattatgggatctggtggctggaaaaacaagagtgacattaacaaatcacaaaaaatcagttagggctgtggttttacatccaagacattacacatttgcatctggttctccagataacataaagcagtggaaattccctgatggaagtttcattcaaaatctttccggtcataatgctattattaacacattgacggtaaattctgatggagtgcttgtatctggagctgacaatggcaccatgcatctttgggactggagaactggctacaattttcagagagttcacgcagctgtgcaacctgggtctttggacagtgaatcaggaatatttgcttgtgcttttgatcagtctgaaagtcgattactaacagctgaagctgataaaaccattaaagtatacagagaggatgacacagccacagaagaaactcatccagtcagctggaaaccagaaattatcaagagaaagagattttaatga

SEQ ID NO: 41 - DNA, 1356 bp, Saccharomyces cerevisiae
atggacggaaatgatcacaaagtcgaaaatttaggagatgtagacaaattttattccagaatacgctggaataaccaattttcatatatggccactctgccgccccacctacaaagcgaaatggaaggtcagaaatcattgctaatgcgatatgatacttataggaaggaaagttcttcttttagtggtgaaggcaagaaagttactctgcagcatgttcctacagatttttcagaagcatcacaagcagtgattagcaagaaagatcacgatacgcatgcatctgcttttgtgaataaaattttccaaccagaggttgctgaagaacttatagttaatcgatacgaaaaacttctgtcacaaaggccggaatggcatgcaccctggaaactttcacgcgttatcaatggccatcttggatgggtacgatgcgttgcaatcgatcctgttgacaacgaatggttcatcaccggaagtaatgatacgacaatgaaagtttgggatcttgcaacaggaaaattaaaaactaccttagcagggcatgtaatgacagtgagagacgttgctgtgtcagatcgacatccttatttattttctgttagtgaagataagacggtcaaatgctgggacctagagaaaaaccaaattattagagattactatggacatttatcgggggttcgtacggtgagcatacatccaacgctggatctcatagctaccgcaggccgagatagcgttatcaaactctgggatatgagaaccagaatacctgttattacactagttgggcataagggtccaatcaatcaagtacagtgtactccagtagaccctcaagtggtgagttcatcgactgatgctacggtaaggttatgggatgtagttgctgggaaaacaatgaaagttctaacacatcataagaggtctgtgagagctacagcgttgcatcctaaggagttttcggtggcttctgcgtgtactgatgacatcagatcatggggattagcagaggggtctttactcaccaattttgagtctgaaaagacaggcataatcaatactttaagcattaatcaagatgatgtattattcgctggcggtgacaatggtgtgctttccttttatgattataagtctggtcacaaataccaatcgttggccacgagagaaatggtaggctctctggaaggtgaacggagtgttctttgtagcactttcgataaaacaggtttaagattaatcactggagaagcagacaaaagcataaagatttggaaacaggatgagacggctacaaaagagtcagaaccggggctagcgtggaaccccaacttaagcgccaaaagattttag

SEQ ID NO: 42 - DNA, 2093 bp, Schizosaccharomyces pombe
atgacagaagcaaaaaacatatcggatgatacagatgtactttctttgactacaaatagtcttagaacatcaaaaactctttttggagccgagtttgggtcagttaccagttttgacgatactgtagctcaaaatctcaaaagaacatacaaagaacatttagaatatggttctgttcttggaggaactgttggaaaacggaagaaccgacattacgaagaagacactattggtagtaatgctttgacggttagagcagacagtgaaaacccttcctcccaagtgatcaccaagttttcagatcctaataagaaaatagcagggcaggtttccatgcaatctttagaaaaaattaaaggagttcctgaagcagcacatagaattgccggagaatcccaggcttctctcgtaaagcgcactcttgccgaacagattcgccctgaatggcatgctccatggactcttatgcgtgttattagtggtcatttgggttgggttcgatgcgtagatgttgaacccggtaatcaatggttttgcacgggtgctggggaccgcaccatcaaaatatgggacttagcgtctggagtacttaagctaacattgactgggcatatcgccactgtgcgaggattagcagtatctccccgtcatccatatttattttcctgtggtgaagacaaaatggtaaaatgctgggacctcgaaactaataaagtgatccgccattatcacgggcatctttccggagtctatgccttaaaactccatccaacacttgacgtccttgttaccgctggtcgtgatgcagtcgctcgtgtttgggatatgcgcactcgccaaaatgtccatgttttatcgggccacaaatccactgttgcttctttagccgttcaagaattcgatcctcaagtagtcacgggttccatggattctactatcagattatgggatttagcagctgggaaaaccttgacgaccttgacacatcacaaaaaaactgttcgtgccctgtctttgcatccagatgaatttacttttgccagtggctcgtccgacaatatcaaacattggaaattccctgaaggcgcatttatgggtaattttgaaggacataatgcaatcgttaatactctttcaataaactcagataatgttatgttttctggtgctgataatggaagcatgtgcttttgggattggaagtctggtcacaaatatcaagagcttcaatctgttgttcaacctggttcattggatagcgaagctggaatctttgctagttcttttgataaaaccggtttgcgtctaattacttgtgaggctgataaaagcgtaaagatatacaaacaagtggacaatgccacacctgaaacacatcccaatttgccatggacaccatcaaatcttcgtcggcgatattaatgatttcatcggacgccattaattatccgtttggaaattcaaaagtgaaaggaacggctaacaaagtgcctgatttgtcaaatgaagaaattgagaggtgtagattgttactaaagaaagagatagggcaactagaaagtgacgattatatccaattcgagaaggaatttttggaaacgtacagtgcacttcacaacacctcaagtttgttgccaggcttggtaatctacgaggaagacgatgaagacgttgaagccgctgaaaagttttatacgaacgacattcaacgagatttagctaagaaagcattggaatgtaacaagttggaaaatcgggtttatgatttggttagatcttcatatgagcaacgtaattttttgataaagaaaatctcgcatgcttggaaggctttgcaaacagaaaggaaaaatttaacgtgctacgaatttctatacaatcaggagcgattagctctacctaataggcttgaagcagctgaaatagagctaagcaaaatgcaacaaatcgaggcgtatgctcaacaagattatgctagggttactggacaaaattaaacttcacttcactttgttttaaataattgtcatagcttatcattcatagtgtatattatcatgcctgaaataaaacgaaagttttataaaaagata

SEQ ID NO: 43 - protein, 23 aa, Homo sapiens
CSRDSTARIWDVRTKASVHTLSG

(19) United States
(12) Patent Application Publication
(10) Pub. No.: US 2004/0253646 A1
(43) Pub. Date: Dec. 16, 2004

(54) Pre-mrna splicing screening assay

(76) Inventors: Angus Iain Lamond, Wormit (GB); Paul Ajuh Munya, Broughty Ferry (GB)

Correspondence Address: MYERS BIGEL SIBLEY & SAJOVEC, PO BOX 37428, RALEIGH, NC, 27627, US

(21) Appl. No.: 10/478,197

(22) PCT Filed: May 20, 2002
(86) PCT No.: PCT/GB02/02393

(30) Foreign Application Priority Data
May 19, 2001 (GB) 0112262.1

(51) Int. Cl. G01N 33/53; G01N 33/567

(52) U.S. Cl. 435/7.2

(57) ABSTRACT

The present invention relates to a method for screening agents which modulate the binding of CDC5L to PLRG1, or specified fragments thereof, and agents identified using such an assay. The present invention also provides small peptides capable of inhibiting pre-mRNA splicing. Such agents are candidates for use in the treatment of, for example, cancer, or fungal infections.

The present invention relates to a method for screening agents which modulate the binding of CDC5L to PLRG1, or specified fragments thereof, and agents identified using such an assay. The present invention also provides small peptides capable of inhibiting pre-mRNA splicing. Such agents are candidates for use in the treatment of, for example, cancer, or fungal infections.

Pre-mRNA splicing occurs in the eukaryotic cell nucleus through a process that involves the removal of non-coding sequences (introns) in the pre-mRNA and the joining of adjacent or alternate coding sequences (exons) to produce mature mRNA. The splicing reaction takes place via a two-step transesterification mechanism involving nucleophilic attacks of phosphodiester bonds and creation of new bonds resulting in the formation of mature mRNA that is then exported to the cytoplasm for use in protein synthesis (for review see Staley and Guthrie, 1998). Splicing is catalysed by a large ribonucleoprotein complex made up of over a hundred proteins called the spliceosome. The spliceosome complex contains four ribonucleoprotein (snRNP) particles (U1, U2, U5, and U4/U6), each of which contains the corresponding snRNA and a set of specific and common proteins (Kramer, 1995; Kramer, 1996; Will and Lurhmann, 1997). The complex also contains multiple non-snRNP associated proteins that are essential for spliceosome assembly and catalysis (Will and Lurhmann, 1997; Staley and Guthrie, 1998). Spliceosome assembly involves the sequential association of the snRNP particles and other proteins onto the pre-mRNA substrate prior to catalysis (reviewed in Reed and Palandjian, 1997).

Several recent studies have suggested that both the human proteins CDC5L and PLRG1 and their respective homolgues in yeast are associated with pre-mRNA splicing factors (Neubauer et al., 1998; Tsai et al., 1999; McDonald et al., 1999; Burns et al., 1999; Ajuh et al, 2000). The human proteins' possible involvement in pre-mRNA splicing was first suggested when both proteins were identified by mass spectrometry in purified spliceosomes assembled on adeno-pre-mRNA in vitro (Neubauer et al., 1998). However, other cellular roles for CDC5L have also been proposed; for example, it has been suggested that CDC5L may also be involved in transcription because of sequence similarities in the amino terminus domain of the protein with the proto-oncogenic transcription factor, c-Myb (Ohi et al., 1994; Bernstein and Coughlin, 1997). Also, the A. thaliana homologue of this protein (AtCDC5), when over expressed in S. pombe, can complement a growth defective phenotype of an S. pombe CDC5+ temperature-sensitive mutant. A sequence-specific DNA binding activity has been reported for AtCDC5 (Hiriyama and Shinozaki, 1996). A possible role for this protein in the regulation of the cell division cycle was observed in a genetic screen of S. pombe for cell division cycle mutants. The study indicated that the CDC5+ gene encodes an essential protein and that the gene's function may be necessary in the G2 phase of the cell cycle (Ohi et al., 1994). More recently, it has been shown that over-expression of CDC5L in mammalian cells shortened the G2 phase of the cell cycle. Also, a dominant negative mutant of the protein lacking the carboxyl-terminal activation domain slowed G2 progression and delayed entry into mitosis (Bernstein and Coughlin, 1998).

The human protein PLRG1, recently identified as a component of the CDC5L complex, is highly homologous to the Arabidopsis thaliana PRL1 gene product. The PRL1 protein has been shown to be essential for the regulation of glucose and hormone responses in Arabidopsis thaliana (Nemeth et al., 1998). Mutations in PRL1 have pleiotropic phenotypes. For example, a prl1 mutation can cause transcriptional de-repression of glucose responsive genes; augment the sensitivity of the plants to growth hormones such as cytokinin, abscisic acid, ethylene and auxin; stimulate the accumulation of sugars and starch in the plants' leaves and inhibit root elongation (Nemeth et al., 1998). In both A. thaliana and COS-1 cells, PRL1 shows nuclear localisation and interacts with ATHKAP2, an α-importin nuclear import receptor (Nemeth et al., 1998). PLRG1 and the PRL1 protein both contain seven copies each of the phylogenetically conserved WD-repeat domains that were first characterised in beta transducin (Neer et al., 1994). Proteins with WD domains are thought to have regulatory functions in the cell as well as being involved in protein-protein interactions. These WD domain proteins have been identified in a variety of species, from human to the facultatively thermophilic actinomycete—Thermospora curvata (Janda et al., 1996; Smith et al., 1999). in a phenotypic screen for cell cycle mutants of an A. thaliana cDNA library in fission yeast, PRL1 was identified as one of 11 genes that can cause severe morphological changes in the yeast. This was interpreted to indicate that PRL1 may be involved in cell shape maintenance and/or regulation of the cell cycle (Xia et al., 1996). More recently, it has been shown that mutations in the PLRG1 homologue in Saccharomyces pombe: prp5⁺ result in defects in pre-mRNA splicing and also blocks progression of the cell division cycle at the G2/M phase (Potashkin et al., 1998).

As mentioned above, several recent studies have shown that CDC5L and PLRG1 are associated with pre-mRNA splicing factors (Neubauer et al., 1998; Tsai et al., 1999; McDonald et al., 1999; Burns et al., 1999; Ajuh et al., 2000). In yeast it has been demonstrated that cells lacking cdc5+ function are defective in pre-mRNA splicing (McDonald et al., 1999). The CDC5L gene product is highly conserved across species and homologues have been identified in several eukaryotic species including Saccharomyces pombe, Sacchammyces cerevisiae, C. elegans, D melanogaster and Xenopus laevis (Hiriyama and Shinozaki, 1996; Stukenberg et al., 1997; Ohi et al., 1998; Tsai et al, 1999). Like CDC5L, PLRG1 is also very highly conserved across species (Nemeth et al., 1998), suggesting essential cellular functions for these proteins. However, a direct role for PLRG1 in the catalytic steps of pre-mRNA splicing has not been shown in spite of its association with splicing factors.

A protein complex associated with CDC5L from HeLa nuclear extracts was recently purified and shown that this complex contains at least six “core” proteins (Ajuh et al., 2000). Two of the core proteins, which also are the most highly conserved across species, are CDC5L and PLRG1.

The present invention is based in part on the results of studies into the interaction of CDC5L and PRLG1 and the observation that CDC5L and PLRG1 interact directly in vitro.

It is amongst the objects of the present invention to provide a method of screening candidate agents for any modulatory effects on the binding of CDC5L to PLRG1.

It is also an object of the present invention to provide peptides capable of inhibiting pre-mRNA splicing.

### SUMMARY OF THE INVENTION

In a first aspect the present invention provides a method for identifying a substance capable of modulating an interaction between (i) a CDC5L polypeptide or a homologue thereof, or a derivative thereof, and (ii) a PLRG1 polypeptide, or a homologue thereof, or a derivative thereof, which method comprises:

a) providing a CDC5L polypeptide or a homologue, or a derivative thereof, as a first component and a PLRG1 polypeptide or a homologue, or a derivative thereof, as a second component;

b) contacting the two components with a test substance under conditions that would permit the two components to interact in the absence of said test substance; and

c) determining whether said substance modulates the interaction between the first and second components.

The method may further comprise

d) administering a substance which has been determined to disrupt the interaction between the first and second components to a eukaryotic cell; and

e) determining the effect of the substance on the cell.

It is understood that the term “modulation” refers to both positive and negative modulation. “Positive modulation”, as used herein refers to an increase in the interaction such as binding of CDC5L polypeptide or a homologue thereof, or a derivative thereof to PLRG1 or a homologue thereof, or a derivative thereof relative to the level of binding and/or activity as a result of the binding in the absence of the substance. “Negative modulation” as used herein refers to a decrease in the interaction such as binding of CDC5L polypeptide or a homologue thereof, or a derivative thereof to PLRG1 or a homologue thereof, or a derivative thereof relative to the level of binding and/or activity as a result of the binding in the absence of the substance.

The invention further provides a substance capable of modulating an interaction between (i) a CDC5L polypeptide or a homologue thereof, or a derivative thereof, and (ii) PLRG1 or a homologue thereof, or a derivative thereof, for use in treating the human or animal body by therapy or for use in diagnosis, whether or not practised on the human or animal body. Such a substance may thus be used in the prevention or treatment of for example cancer, or fungal infections. Such a substance may be for example, a peptide fragment of CDC5L or PLRG1 designed to act competitively with the native CDC5L or PLRG1 and thereby prevent or reduce binding of CDC5L to PLRG1. This in turn is envisaged to prevent or reduce pre-mRNA splicing.

The invention therefore further provides a substance capable of modulating an interaction between (i) a CDC5L polypeptide, or a homologue thereof, or a derivative thereof, and (ii) PLRG1 or homologues thereof, or derivatives thereof, for use in regulating mRNA splicing and hence the cell cycle of a mammalian, yeast or fungal cell. The substance may be used for inhibiting protein synthesis by disrupting mRNA splicing and is therefore applicable to the treatment of diseases in which undesirable proliferation of cells or disease-causing agents occurs. In that event, the cell may for example be a tumour, yeast or fungal cell, or any other disease-causing organism which possesses or utilises CDC5L and PLRG1 or homologues, such as viruses, parasites and skin diseases, such as psoriasis.

The invention also provides a method of regulating and/or disrupting the cell cycle in a eukaryotic cell, which method comprises administering to said cell a substance capable of modulating an interaction between (i) a CDC5L polypeptide or a homologue thereof, or a derivative thereof, and (ii) PLRG1 or a homologue thereof, or a derivative thereof.

### DETAILED DESCRIPTION OF THE INVENTION

Polypeptide Components

The first component comprises a CDC5L polypeptide or a homologue thereof or a derivative of CDC5L or of a CDC5L homologue. In particular, the inventors have identified a portion of the carboxy terminus region of CDC5L as being capable of interacting with PLRG1, specifically a region encompassing amino acid residues 602 to 800 of the human sequence (numbering according to that shown in FIG. 9a) and especially amino acid residues 706 to 800. It is postulated that the exact region responsible for the interaction with PLRG1 resides within residues 706 to 800, and therefore, may be smaller than this defined region. It is well within the skill of the person in the art to be able to identify the smaller regions by, for example, synthesising smaller regions comprising residues between 706 to 800 of CDC5L and examining their interaction with PLRG1AII amino acid residue numbers discussed herein are as shown in FIGS. 9(a) and 9(c) of the polypeptide sequences.

Therefore, the present invention provides a CDC5L polypeptide, homologue, fragment or derivative thereof. The fragments may be greater than 50, 60, 100 or 200 amino acid residues long. The minimum fragment length may be 80, 100 or 120 amino acid residues and may be 94 amino acid residues, which are capable of binding to PLRG1. Such fragments include fragments containing a portion of the carboxy terminal region of CDC5L, typically a region of CDC5L encompasssing amino acid residues 602 to 800, and preferably amino acid residues 706 to 800. It is thought that the exact region responsible for the interaction with PLRG1 resides within residues 706 to 800, while residues 602 to 706 appear to enhance the interaction between the polypeptides. Herein, substantial homology for fragments of CDC5L is regarded as a sequence which has at least 70%, eg. 80%, 90%, 95% or 98%, amino acid homology (identity) over at least 50, preferably 75, more preferably 100 amino acids with a portion of the carboxyl terminus of CDC5L.

Derivatives further include variants of CDC5L and its homologues or derivatives, including naturally occurring allelic variants and synthetic variants which are substantially homologous to said CDC5L and its homologues. The human CDC5L sequence may, for example, be found in the SwissProt database as accession number Q99974, and the yeast equivalent CeF1 sequence may, for example, be found in the SwissProt database as accession number NP_{—}013940.

The second component is selected from PLRG1 or homologues thereof, and their derivatives (derivatives are defined as for CDC5L). In addition to CDC5L, the present inventors have also identified a WD motif of PLRG1 which is capable of interacting with CDC5L, specifically amino acid residues 257 to 396. It is thought that the exact region responsible for the interaction with CDC5L resides within residues 257 to 396, and therefore, may be smaller than this defined region. It is also well within the skill of the person in the art to be able to identify the smaller regions, by, for example, synthesising smaller regions encompassed by residues 257 to 396 and examining their interaction with CDC5L.

The human PLRG1 sequence may, for example, be found in the Swiss Prot databasE as accession number O43660, and the S. pombe equivalent, PRP46, may for example, be found in the SwissProt database as accession number AAG01399, and the S. cerevisiae equivalent may, for example, be found in the SwissProt database as accession number NP_{—}015174.

Therefore, the present invention also provides a PLRG1 polypeptide or derivative thereof. Derivatives of PLRG1 include fragments, comprising at least 100, 140 or 160 amino acids, such as at least 130 amino acids, and for example 139 amino acids, which are capable of binding to CDC5L. Derivatives further include variants of PLRG1, its homologues or derivatives, for example, WD motifs found in other species, including naturally occurring allelic variants and synthetic variants which are substantially homologous to said PLRG1. In this context, substantial homology is regarded as a sequence which has at least 70%, eg. 80%, 90%, 95% or 98% amino acid homology (identity) over at least 80, such as 100, for example 140 amino acids with PLRG1. The WD motif (WD/40) is described in Neer et al (1994).

It will be understood that for the particular polypeptides embraced herein, natural variations such as may occur due to polymorphisms, can exist between individuals or between members of the family. These variations may be demonstrated by (an) amino acid difference (s) in the overall sequence or by deletions, substitutions, insertions, inversions or additions of (an) amino acid(s) in said sequence. All such derivatives showing the recognised modulatory activity are included within the scope of the invention. For example, for the purpose of the present invention conservative replacements may be made between amino acids within the following groups:

(I) Alanine, serine, threonine;

(II) Glutamic acid and aspartic acid;

(III) Arginine and leucine;

(IV) Asparagine and glutamine;

(V) Isoleucine, leucine and valine;

(VI) Phenylalanine, tyrosine and tryptophan.

Derivatives may be in the form of a fusion protein wherein CDC5L and/or PLRG1, a homologue or derivative thereof is fused, using standard cloning techniques, to another polypeptide which may, for example, comprise a DNA-binding domain, a transcriptional activation domain or a ligand suitable for affinity purification (for example glutathione-S-transferase or six consecutive histidine residues).

The first and second components used in the assays may be obtained from for example mammalian, yeast or fungal extracts, produced recombinantly from, for example, bacteria, yeast or higher eukaryotic cells including mammalian cell lines and insect cell lines, or synthesised de novo using commercially available synthesisers. Preferably, the first and second components used in the assays are recombinant.

Therefore, the present invention provides an assay system comprising CDC5L polypeptide, or derivatives thereof, and PLRG1 polypeptide, or derivatives thereof. Preferably, the assay comprises a derivative comprising a portion of the carboxy terminal region of CDC5L and a derivative comprising the WD motif region or fragment thereof PLRG1. More preferably, the assay comprises a derivative comprising a region encompassing amino acid residues 602 to 800, in particular amino acid residues 706 to 800, of CDC5L, and a derivative comprising amino acid residues 257 to 396 of PLRG1. The smaller regions are preferable due to their small size, resulting in potentially reduced complications in the production and handling of them.

Further, the present invention provides an isolated nucleic acid for the recombinant production of CDC5L, as disclosed in FIG. 10(a) or 10(b) or derivatives thereof, and an isolated nucleic acid for the recombinant production of PLRG1 as disclosed in FIG. 10(c), or derivatives thereof.

The invention still further provides a nucleotide sequence which is similar to the disclosed DNA sequences. By “similar” is meant a sequence which is capable of hybridising to a sequence which is complementary to the inventive nucleotide sequence. When the similar sequence and inventive sequence are double stranded the nucleic acid constituting the similar sequence preferably has a Tₘ within 20° C. of that of the inventive sequence.

In the case that the similar and inventive sequences are mixed together and denatured simultaneously, the Tm values of the sequences are preferably within 10° C. of each other. More preferably hybridization may be performed under stringent conditions, with either the similar or inventive DNA preferably being supported. Thus for example either a denatured similar or inventive sequence is preferably first bound to a support and hybridization may be effected for a specified period of time at a temperature of between 50 and 70° C. in double strength SSC (2×NaCl 17.5 g/l and sodium citrate (SC) at 8.8 g/l) buffered saline containing 0.1% sodium dodecyl sulphate (SDS) followed by rinsing of the support at the same temperature but with a buffer having a reduced SSC concentration. Depending upon the degree of stringency required, and thus the degree of similarity of the sequences, such reduced concentration buffers are typically single strength SSC containing 0.1% SDS, half strength SSC containing 0.1% SDS and one tenth strength SSC containing 0.1% SDS.

Sequences having the greatest degree of similarity are those the hybridization of which is least affected by washing in buffers of reduced concentration. It is most preferred that the similar and inventive sequences are so similar that the hybridization between them is substantially unaffected by washing or incubation at high stringency, for example, in one tenth strength sodium citrate buffer containing 0.1% SDS.

Candidate Substances

A substance which modulates an interaction between the first component and the second component may do so in several ways. It may directly modulate the binding of the two components by, for example, binding to one component and masking or altering the site of interaction with the other component. Candidate substances of this type may conveniently be screened by in vitro binding assays as, for example, described below. Examples of candidate substances include non-functional homologues of the first or second components as well as antibodies which recognise the first or second components.

Suitable candidate substances include for example peptides, especially of from about 10-15 to 100 amino acids in size, in particular 10 to 20 to 30 amino acids in size, based on the sequence of a region of CDC5L encompassing amino acid residues 706 to 800, or variants of such peptides in which one or more residues have been substituted, and peptides based on the sequence of a region of PLRG1 encompassing amino acid residues 257 to 396, or variants of such peptides in which one or more residues have been substituted.

It has been observed that the peptides

NH₂-PEDTVDFLKEVESRMQHITQGRTSMKIQFK-COOH
NH₂-PPTEVLLESIQSKVESIEQLQRKLQHVQ-COOH
NH₂-EQQNNEMCSTLCHHSLPALIEG-COOH
NH₂-HHSLPALIEGQRKYYADYYAYRQEI-COOH

Disrupt the interaction between CeF1 and PRP46 polypeptides (equivalent to human CDC5L and PLRG1, respectively) in yeast (S. cerevisiae).

Suitable candidate substances also include antibody products (for example, monoclonal and polyclonal antibodies, single chain antibodies, chimeric antibodies and CDR-grated antibodies) which are specific for the first component or the second component, preferably to residues 706 to 800 of CDC5L, or to residues 257 to 396 of PLRG1.

Furthermore, combinatorial libraries, peptide and peptide mimetics, defined chemical entities, oligonucleotides, and natural product libraries may be screened for activity as inhibitors of an interaction between the first component and the second component in assays such as those described below. The candidate substances may be used in an initial screen in batches of, for example 10 substances per reaction, and the substances of those batches which show inhibition tested individually. Candidate substances which show activity in in vitro screens such as those described below can then be tested in in vivo systems, such as mammalian, yeast or fungal cells which will be exposed to the inhibitor and tested for susceptibility to cell cycle disruption.

Assays

The assays of the invention may be in vitro assays or in vivo assays, for example using an animal, yeast or fungal model. One type of in vitro assay for identifying substances which disrupt an interaction between the first component and the second component involves:

contacting a first component, which is immobilised on a solid support, with a non-immobilised second component in the absence of a candidate substance;

contacting the first immobilised component with the non-immobilised second component in the presence of a candidate substance; and

determining if the candidate substance disrupts the interaction between the first component and the second component.

Alternatively, the second component may be immobilised and first component non-immobilised.

Binding of the first component to the second component (and vice-versa) may be determined by a variety of methods well-known in the art. For example, the non-immobilised component may be labelled (with for example, a radioactive label, an epitope tag or an enzyme-antibody conjugate). The effect of a candidate substance on an interaction between the two components can be determined by comparing the amount of label bound in the presence of the candidate substance with the amount of label bound in the absence of candidate substance. A lower amount of label bound in the presence of the candidate substance indicates that the candidate substance is an inhibitor of interactions between the first component and the second component.

Alternatively, binding may be determined by immunological detection techniques. For example, the reaction mixture can be Western blotted and the blot probed with an antibody that detects the non-immobilised component. ELISA techniques may also be used.

Candidate substances that are identifiable by the method of the invention as modulating an interaction between a first component and a second component may be tested for their ability to, for example, disrupt mRNA splicing, which may lead to inhibition of protein synthesis. Such compounds could be used therapeutically in regulating and/or disrupting the cell cycle of a mammalian, yeast or fungal cell, including, for example, inducing cell death in, for example, neoplastic cells, or preventing cell growth in yeast or fungal cells.

The present inventors have in fact identified peptides based on the human sequence of the CDC5L protein sequence between residues 706-740 and a corresponding region from other species (see FIG. 9a) which have been shown to inhibit pre-mRNA splicing.

More particularly the present inventors have found the region comprising the sequence EKKMKILLGGYQ (amino acid nos. 714-725) of the human sequence and corresponding sequences from other species to be able to inhibit pre-mRNA splicing. Additionally, each peptide appears to be generally species specific. That is, a peptide based on the corresponding region of for example Plasmodium falciparum has a significantly reduced effect on pre-mRNA splicing in human cells.

Thus, in a further aspect the present invention provides a peptide comprising the sequence EKKMKILLGGYQ of the human CDC5L polypeptide or corresponding region from a homologous sequence of a different species for use in inhibiting or reducing pre-mRNA splicing.

Corresponding regions from a homologous sequence of a different species may easily be determined by a skilled addressee and are shown for example in FIG. 9a. Suitable homologous sequences include EKKLKILTGGYZ from Drosophila melanogaster; EKKLGKVLGGYD from Lentinula (fungi); and ENKYDIYTKGYQ from Plasmodium falciparum.

As mentioned above, the present inventors have observed that the peptides are generally quite species specific. Thus, it is envisaged that the Lentinula peptide mentioned above could for example be used to treat fungal infections in humans, such that the peptide would inhibit fungal pre-mRNA splicing but not substantially effect human pre-mRNA splicing.

In addition to the CDC5L derived peptides described above which have been shown to disrupt pre-mRNA splicing, the present inventors have also identified peptides between positions 257-396 of the human PLRG1 sequence which can also inhibit pre-mRNA splicing. The following sequences have been shown to inhibit pre-mRNA splicing:

PYLFSCCEDKQVKCWDLEYNKVIRHYHGHL
and
PQIITGSHDTTIRLWDLVAGKTRVTLTNHK.

Typically, an assay to determine the effect of a candidate substance identifiable by the method of the invention on the disruption of mRNA splicing in a mammalian, yeast or fungal cell comprises:

(a) administering the candidate substance to the cell; and

(b) determining the effect of the candidate substance on the cell cycle, including, for example the prevention of mRNA splicing which leads an inhibition of protein synthesis.

Administration of candidate substances to cells may be performed by for example adding directly to the cell culture medium or injection into the cell. The assay is typically carried out in vitro. The candidate substance is contacted with the cells, typically cells in culture. The cells may be cells of a mammalian, yeast or fungal cell line.

The ability of a candidate substance to reduce or prevent protein synthesis can be determined by administering a candidate compound to cells and determining if protein synthesis has been reduced in said cells. The effect of the disruption of protein synthesis can be measured indirectly by measuring the amount of spliced pre-mRNA in the cell after administration of the reagent or directly by measuring the incorporation of amino acids that have been radioactively, fluorescently labelled or any other tagged amino acids into newly synthesised proteins in cells exposed to the reagent. The tagged or labelled amino acids could be injected into cells or included in the growth medium after administration of the CDC5L reagent. Inhibition of protein synthesis is measured as the decrease in incorporation of labelled amino acids into proteins or by measuring the level of unspliced message i.e. accumulation of pre-mRNA due to splicing inhibition, or an absence of mature mRNA.

Therapeutic Uses

It has been demonstrated herein that mRNA splicing can be disrupted in HeLa cells. Whilst inhibition of splicing results in inhibition of de novo protein synthesis, protein already present in the cells remains intact. Not intending to be bound by theory, it is postulated that since new proteins, and hence protein synthesis, are required to complete a cell cycle in rapidly dividing cells, cells in which proliferation is undesirable, for example tumour cells, may be selectively targetted with agents which disrupt the interaction between CDC5L and PLRG1. It is thought that failure to complete the cell cycle in these cells may arrest tumour growth or even induce apoptosis.

Furthermore, although CDC5L and PLRG1 are also involved in mRNA splicing in yeast and fungal cells, it is postulated that differences between the human and yeast, and human and fungal proteins may be the target of agents which disrupt the interaction between CDC5L and PLRG1 (see FIG. 8). Hence, selecting for agents which disrupt only the yeast and fungal CDC5L and PLRG1 interaction may provide a selective treatment against these organisms.

Thus, the present invention provides a substance capable of modulating an interaction between (i) a CDC5L polypeptide or a homologue thereof, or a derivative thereof, and (ii) PLRG1 or homologues thereof, or derivatives thereof, for use in a method of disrupting mRNA splicing and hence substantially reducing protein synthesis. Typically, said substance may be used to target diseases in which cell proliferation is undesirable and may be used to induce cell death, for example in a tumour cell, or to prevent cell growth, in for example a tumour cell, a yeast or fungal cell(s).

The formulation of a substance according to the invention will depend upon the nature of the substance identified but typically a substance may be formulated for clinical use with a pharmaceutically acceptable carrier or diluent. For example it may be formulated for topical, parenteral, intravenous, intramuscular, subcutaneous, intraocular or transdermal administration. A physician will be able to determine the required route of administration for any particular patient and condition.

Typically, the substance is used in an injectable form. It may therefore be mixed with any vehicle which is pharmaceutically acceptable for an injectable formulation, preferably for a direct injection at the site to be treated. The pharmaceutically-acceptable carrier or diluent may be, for example, sterile or isotonic solutions. It is also preferred to formulate that substance in an orally active form.

The dose of substance used may be adjusted according to various parameters, especially according to the substance used, the age, weight and condition of the patient to be treated, the mode of administration used and the required clinical regimen. A physician will be able to determine the required route of administration and dosage for any particular patient and condition.

The present invention will now be further described by way of example and with reference to the Figures which show:

### FIGURE LEGENDS

FIG. 1 illustrates that anti-CDC5L co-immunodepletes most of the PLRG1 from HeLa nuclear extract. Anti-CDC5L was used to immunodeplete CDC5L protein from nuclear extract. The beads containing immunoprecipitated proteins and supernatants from which CDC5L has been removed were loaded onto a 10% SDS-PAGE gel and probed by Western blotting using anti-CDC5L antibodies. Lanes marked 1 are the control lanes containing about 50 μg of HeLa nuclear extract and the lanes labelled 2 have the same amount of nuclear extract as lanes 1 except that the extract in lanes 2 was collected from an immunodepletion experiment using pre-immune IgG. The lanes marked 3 and 4 contained proteins eluted from protein G agarose beads used in the immunodepletion of CDC5L from nuclear extract by pre-immune IgG and anti-CDC5L antibodies respectively. The supernatant from the immunodepletion experiment using anti-CDC5L antibodies was loaded into the lanes labelled 5 of the figure. Immunoprecipitation reactions in panel A were probed with anti-CDC5L antibodies. Panels B and C contained identical samples as in A except that B and C were probed with anti-PLRG1 and anti-SPF30 antibodies respectively.

FIG. 2 illustrates that CDC5L co-localises with PLRG1 in vivo in human HeLa cells. HeLa cells were grown, transfected, and incubated with antibodies as described in the Materials and Methods section. The images shown in the panels are representative optical sections from the respective deconvolved data sets. All the panels marked (i) show antibody staining while the panels marked (ii) indicate the nuclear structures obtained using GFP-CDC5L or GFP-PLRG1. All the panels labeled (iii) show overlays of (i) and (ii) in each experiment.

(A) HeLa cells were transiently transfected with a plasmid expression vector encoding the GFP-PLRG1 fusion protein, fixed and stained with the anti-Sm protein monoclonal antibody Y12 (Petterson et al., 1984). Red represents Y12 staining and green indicates GFP-PLRG1 expression.

(B) A plasmid expression vector encoding GFP-CDC5L was used to transiently transfect HeLa cells that were subsequently fixed and stained with the same anti-Sm antibody as above. Red indicates Y12 staining as above while green here represents GFP-CDC5L expression.

(C) HeLa cells were transfected with the GFP-CDC5L plasmid expression vector as above. The cells were then fixed and stained with anti-PLRG1. Red represents anti-PLRG1 staining and green shows GFP-CDC5L expression. In all the panels, yellow indicates co-localisation of the two proteins CDC5L and PLRG1. Similar results were obtained using the protein specific antibodies instead of the GFP fusions (data not shown). Bar, 10 μm.

FIG. 3 illustrates the interaction between CDC5L and PLRG1 in vitro.

(A) GST-PLRG1 (lanes 2 and 3) was incubated for 20 minutes at room temperature with 0.5 mg of HeLa nuclear extract. An immunoprecipitation was performed on the reaction either using control pre-immune serum: CTRL1 (lane 2) or anti-CDC5L antibodies (lane 3). The blotted proteins from the immunprecipitates were probed with anti-PLRG1 antibodies. Lane 4 shows a pull-down experiment in which GST-CDC5L was incubated with nuclear extracts as above and the interacting proteins pulled down using 25-30 μl of glutathione sepharose beads. The blot was probed with anti-GST-PLRG1 antibodies. Lane 5 is a control reaction using GST alone in the pull-down experiment instead of GST-CDC5L. Lane 1 contained approximately 40 μg of HeLa nuclear extract. Note that the anti-GST-PLRG1 anti-serum (which also contains GST antibodies) recognises full-length GST-CDC5L and a minor band in lane 4 corresponding to a partial cleavage product of the GST-CDC5L protein used in the pull-down experiment as well as endogenous PLRG1.

(B) GST-PLRG1 (0.2 nmole) was used to pull down L-[³⁵S] methionine labeled in vitro translated CDC5L (5-8 μL). Lanes 1 and 2 are duplicate samples of the pull-down experiment whereas lanes 3 and 4 are controls using glutathione sepharose beads alone and GST respectively for pull-downs. Lane 5 contained about 30-40% of the input labeled CDC5L.

(C) GST-PLRG1 was used to pull down bacterially expressed His-tagged CDC5L. Lanes 1 and 2 contained duplicate samples of the pull-down of His-CDC5L by GST-PLRG1. Lane 3 had a control pull-down using the spliceosomal protein SPF30 which does not interact with CDC5L. Lane 4 is the positive control and contained His-CDC5L alone used in the binding assays. All the arrows on the right of the panels indicate proteins identified in the pull-down experiments.

FIG. 4 illustrates the identification of the PLRG1 binding domain in CDC5L.

(A) Point mutations were inserted into the CDC5L cDNA creating stop codons that resulted in truncation mutants when the protein is expressed. The arrows indicated the approximate length of expressed protein while the numbers to the right of the arrows show the position of the inserted stop codon. The letters a-f represent the different mutant proteins expressed.

(B) The CDC5L cDNAs with mutations were in vitro translated in the presence of L-[³⁵S] methionine and 3-4 μl of the expressed protein loaded onto a 12% SDS-PAGE gel. Protein bands were revealed by autoradiography. The lanes marked 1 to 6 represent the respective deletion mutants marked a to f. Lane 7 contained full-length CDC5L.

(C) Approximately 0.2 nmole of GST-PLRG1 was used in pull-down experiments with the deletion mutants a-f translated in vitro (8-10 μl). The lane marked G contained the control sample where GST was used to pull-down CDC5L instead of GST-PLRG1. Lanes 1-6 contained the mutant proteins a-f and lane 7 had a pull-down of CDC5L with GST-PLRG1.

(D) Overlapping carboxyl terminal sequences of the CDC5L cDNA were sub-cloned into the expression vector pET-30a (Novagen). The arrows indicate the cloned fragments and the numbers on the left and right-hand side of each arrow represent the start and end of each cDNA fragment. The letters g to j represent the respective sub-clones of the CDC5L cDNA.

(E) The sub-clones above were in vitro translated and 3-5 μL of the translated protein were run on a 12% SDS-PAGE gel. The protein bands were revealed by autoradiography. The lanes marked 2 to 5 contained the expressed proteins from the sub-clones g to j respectively. Lane 1 contained the full-length CDC5L protein.

(F) GST-PLRG1 was used to pull down the proteins from the clones g-j. Lane 1 is a positive control i.e. a pull-down of CDC5L using GST-PLRG1. Lanes 2-5 contained samples from pull-down experiments using GST-PLRG1 and the mutant proteins from g to j respectively.

FIG. 5 illustrates the identification of the CDC5L binding region in PLRG1.

(A) GST tagged PLRG1 cDNA was point mutated such that stop codons were inserted at specific sites. The arrows indicate the length of the reading frames after stop codon insertion. The numbers on the right of the arrows indicate the positions of the stop codons in the PLRG1 cDNA sequence. The letters a to c represent the cDNAs generated by the mutations.

(B) The GST-PLRG1 mutants were expressed in E. coli and the purified proteins used in pull-down assays with CDC5L. Lane 1 is a negative control pull-down using GST whereas lanes 2-4 are pull-downs using the proteins from a to c respectively. Lane 5 represents a positive control experiment where full-length GST-PLRG1 was used to pull-down CDC5L.

(C) Overlapping sequences containing the CDC5L binding region were sub-cloned into the pGEM-T vector (Promega) and pGEX-4-T1 (Pharmacia). The arrows indicate the cloned fragments and the numbers on the left and right-hand side of each arrow represent the start and end of each cDNA fragment. The letters d to f have been used to label the sub-clones produced.

(D) The cDNAs in pGEM-T were used for in vitro transcription/translation and the expressed protein bands revealed by autoradiography. The lanes marked 1-3 represent proteins expressed from the clones d to f respectively.

(E) The proteins expressed above were used in pull-down experiments with GST-CDC5L. Lane 1 is a control experiment in which GST alone was used to pull-down full-length L-[³⁵S] methionine-labelled PLRG1. The lanes marked 2-4 contained pull-downs using GST-CDC5L and the proteins expressed using the clones d to f.

FIG. 6 illustrates that the WD motif rich region of PLRG1 interacts with the carboxyl terminal of CDC5L.

(A) GST-ΔPLRG1f (PLRG1 mutant from clone f) was used to pull-down the carboxyl terminal truncated proteins of CDC5L i.e. ΔCDC5Lg and ΔCDC5Lh. Lane 1 is a control pull-down of CDC5L using GST alone. Lane 2 is a pull-down of CDC5L using full-length GST-PLRG1. Lanes 3 and 4 contained samples from experiments where GST-ΔPLRG1f was to pull down L-[³⁵S] methionine labelled ΔCDC5Lg and ΔCDC5Lh respectively.

(B) GST-ΔPLRG1f, ΔCDC5Lg and ΔCDC5Lj were expressed in E. coli and purified as described in the Materials and Methods section. GST-ΔPLRG1f was used to pull down ΔCDC5Lg or ΔCDC5Lj on to glutathione sepharose beads. All the reactions were carried out in duplicate. Lanes 1 and 2 contained reactions from a pull-down experiment using, ΔCDC5Lg and lanes 3 and 4 contained samples from a pull-down of hexahistidine ΔCDC5Lj using GST-ΔPLRG1f. Lanes 5 and 6 represent control pull-down experiments using GST and ΔCDC5Lg. Note that GST-ΔPLRG1f did not bind to the glutathione sepharose beads as efficiently as GST alone.

(C) Panel C is identical to B except that the nitrocellulose filter was probed with a protein-S alkaline phosphatase conjugate (Novagen).

FIG. 7 illustrates that the interaction between CDC5L and PLRG1 in nuclear extract is required for splicing catalysis.

(A) ΔCDC5Lh or ΔPLRG1f (approximately 1 nmole) was added to approximately 100 μg of HeLa nuclear extract followed by immunoprecipitation using about 15-20 μg rabbit anti-CDC5L antibodies. The immunoprecipitates were subsequently probed with sheep polyclonal anti-CDC5L and anti-PLRG1 antibodies. Lane 1 contained nuclear extract alone. The lanes marked 2, 3, 6 and 7 contained immunoprecipitates of anti-CDC5L except that ΔCDC5Lh alone was added to the nuclear extract in lane 3 and ΔPLRG1f was added to the extract in lane 6 while ΔCDC5Lh and GST-ΔPLRG1f were both added to the nuclear extract in lane 7. Lanes 4 and 5 contained negative control immunoprecipitation reactions using antibodies to HCF (a nuclear protein) and rabbit pre-immune IgG. The panels labelled i and ii were probed with anti-CDC5L and anti-PLRG1 antibodies respectively. The arrows on the right of the figure indicate the protein bands.

(B) Full-length and truncated proteins to be added to splicing reactions were expressed in E. coli and purified to the same extent by affinity chromatography. 2-10 μg of the purified proteins were run on a 4-12% gradient gel (Novex) and stained using colloidal coomassie stain according to the manufacturer's instructions. Lane 1 contained GST. Lanes 2 and 3 had ΔCDC5Lh and GST-ΔPLRG1f respectively. Lane 4 contained GST-PLRG1. The lane marked 5 contained SPF30 while lane 6 contained His-CDC5L. Minor bands in the gel represent degradation products of the expressed proteins.

(C) 0.05 to 0.2 nmoles of the bacterially expressed proteins were added to about 55 μg of HeLa nuclear extract and pre-incubated at room temperature for about 20 minutes before addition to pre-mRNA splicing reactions. The splicing reactions were incubated at 30° C. for about 90 minutes. Lane 1 contained the pre-mRNA used in the splicing experiments. Lanes 2 and 3 contained control splicing reactions except that 0.2 nmoles of GST were added to the reaction in lane 3. Lanes 4-6 represent splicing reactions to which increasing amounts of ΔCDC5Lh (0.05 to 0.2 nmoles) were added whereas the reactions in lanes 12-14 contained increasing amounts of GST-ΔPLRG1f. Lanes 7 and 8 contained splicing reactions to which were added increasing amounts (0.1 and 0.2 nmole respectively) of both ΔCDC5Lh and GST-ΔPLRG1f. The reactions in lanes 9, 10 and 11 contained the E. coli expressed proteins CDC5L, SPF30 and PLRG1 respectively.

FIG. 8 illustrates the inhibition of spliceosome assembly by ΔCDC5Lh The panels A-C represent native polyacrylamide/agarose gels used to separate spliceosome complexes. The bands on the gels were revealed by autoradiography. All the lanes marked CTRL1 contained control splicing reactions prepared and left on ice for 60 minutes. The lanes labelled CTRL2 are control reactions to which GST was added. Approximately 0.2 nmoles of bacterially expressed proteins were added to about 55 μg of HeLa nuclear extract and pre-incubated at room temperature for 20 minutes before addition to pre-mRNA splicing reactions as above except that the reactions here were incubated at 30° C. for 1 hour before loading on to native gels.

(A) Lane 1 contained a splicing reaction prepared and left on ice. Lane 2 is a control reaction to which was added GST. Lanes 3 and 4 contained reactions with had the ΔCDC5Lh and GST-ΔPLRG proteins added respectively. Lane 5 (CTRL3) contained a splicing reaction to which was added both ΔCDC5Lh and GST-ΔPLRG1f proteins. The two proteins had been pre-incubated together for 10 minutes at room temperature before addition into the splicing reactions.

(B) ΔCDC5Lh was added to splicing reactions at different time points. The time point—20 indicates that ΔCDC5Lh was pre-incubated with nuclear extract at room temperature for 20 minutes before the splicing reaction was started. The time points 0, 20, 40 and 60 correspond to the respective times in minutes at which ΔCDC5Lh was added to the splicing reactions after they had been started. All the reactions were allowed to run at 30° C. for up to 60 minutes after addition of the ΔCDC5Lh protein. Lanes 3-7 contain samples representing the different time points.

(C) A single splicing reaction was prepared and incubated for about 50 minutes after which the reaction was split into two aliquots. GST was added to the aliquot in lane 2 and ΔCDC5Lh was added to the aliquot in lane 3. The reactions were then allowed to continue for a further 10-15 minutes before stopping. Spliceosomal complexes were then separated on a native gel.

FIG. 9 illustrates amino acid alignments of (a) CDC5L from S. cerevisiae, S. pombe, A. thaliana, C. elegans, Drosophila and humans (b) the carboxyl terminal region of CDC5L from S. cerevisia, S. pombe, A. thaliana, C. elegans, Drosophila and humans, and (c) PLRG1 from yeast, A. thalian, C. elegans and humans. Residues shaded yellow differ from the consensus by 4 distance units, while residues shaded green match the consensus within 4 distance units.

FIG. 10 illustrates polynucleotides acid encoding (a) human CDC5L, (b) S. cerevisiae CEF 1 (equivalent to human CDC5L), (c) S. pombe CDC5+ (equivalent to human CDC5L), (d) human PLRG1, (e) S. cerevisiae Prp46p (equivalent to human PLRG1), and (f) S. pombe Prp5 (equivalent to human PLRG1).

FIG. 11 shows the splicing inhibition by a 24-mer CDC5L peptide from the PLRG1 interaction domain. Lane 1 in the figure contained the pre-mRNA used in the splicing experiment. Lane 2 is a positive control and no peptide was added to this reaction. The reaction in Lane 3 contained 20 nmole of a non-CDC5L peptide whereas the reactions in Lanes 4, 5 and 6 each contained 20 nmole of overlapping peptides (Nos. 1-3) designed from the PLRG1 interacting region of the CDC5L protein. The symbols on the right of the figure represent the pre-mRNA, splicing intermediates and products present in the reaction at the end of the experiment.

FIG. 12 shows the splicing inhibition by a 12-mer peptide designed from human peptide No. 1. The reaction in Lane 1 of this figure is the positive control containing no additional peptide. Lane 2 contained 20 nmole of the inhibitory 24-mer human CDC5L peptide no. 1. The splicing reactions in lanes 3-5 contained 20 nmole each of the 12-mer CDC5L peptides designed from the 24-mer inhibitory peptide. The symbols on the right of the figure represent the pre-mRNA, splicing intermediates and products present in the reaction at the end of the experiment.

FIG. 13 shows the species specific and dose dependent inhibition of pre-mRNA splicing by CDCL related peptides. Lane 1 in this figure contained the pre-mRNA used in the splicing experiment. The reaction in Lane 2 of this figure is the positive control containing no additional peptide. The splicing reactions in Lanes 3-8 contained 3, 6, 9, 12, 15 and 18 nmole of the inhibitory 12-mer human CDC5L peptide no. 5 respectively. Lane 9 contained the 12-mer reverse peptide ie. no. 8. The reactions in lanes 10 to 12 were performed in the presence of 20, 40 and 60 nmole respectively of the fungal (Lentinula) peptide ie. no 11. Splicing reactions in Lanes 13 to 15 were performed in the presence of 20, 40 and 60 nmole respectively of the Plasmodium falciparum peptide ie. no. 12. The symbols on the right of the figure represent the pre-mRNA, splicing intermediates and products present in the reaction at the end of the experiment.

FIG. 14 shows the affect of splicing inhibitors by PLRG1 derived peptide derived from the CDC5L binding domain. The sequences of the peptides used in the experiment are as follows:

PL1 sequence=PYLFSCGEDKQVKCWDLEYNKVIRHYHGHL

PL2 sequence=CSRDSTARIWDVRTKASVHTLSG

PL3 sequence=PQIITGSHDTTIRLWDLVAGKTRVTLTNHK

7, 14 or 21 nmols of each peptide were added to each pre-mRNA splicing reaction.

### EXAMPLES

### Materials and Methods

cDNA Cloning and Sequencing

The PLRG1 and CDC5L cDNAs were cloned from a HeLa cDNA library (Clontech) by PCR. Primers were designed for the amino and carboxyl termini of the proteins using previously deposited sequences for these cDNAs in the GeneBank database (PLRG1 accession no. AF044333 and CDC5L accession no. U86753). The PLRG1 primers contained BamHI and Xho I sites added to the 5′ ends of the 5′ and 3′-end primers respectively. The CDC5L PCR primers contained Sal I sites added to their 5′ ends. The PCR products were purified on a PCR purification column (Qiagen) according to the manufacturer's instructions. Purified PCR products were digested with the appropriate enzymes and cloned using standard methods into the compatible sites of the vectors pGEX-4T1 (Pharmacia), pET-30a (Novagen), pEGFP-C1 (Clontech) and pSG-9M a modified form of the plasmid pSG5 (Green et al., 1988) containing an amino terminus myc tag. The plasmid DNA was sequenced on the Applied Biosytems 377 automated DNA sequencer using the Taq dye terminator cycle sequencing method according to the manufacturer's instructions. pGEX-4T1 and pET-30a were used for expression of recombinant protein in E. coli while the pEGFP-C1 and pSG-9M vectors were used for expression in mammalian cells (HeLa). pET-30a clones were also used for in vitro transcription/translation experiments.

Expression of Recombinant Proteins in E. coli

cDNAs cloned into the pGEX-4T1 vector were used to transform E. coli BL21(DE3). Overnight cultures were grown from single colonies then diluted 1:10 in fresh LB medium with ampicillin (100 μg/ml) and grown at 30° C. to an OD₆₀₀ of 0.7-1.0 before induction with 0.1 mM IPTG. Three hours post induction, cells were pelleted and resuspended in 10 ml of PBS/0.5% Triton X-100 containing protease inhibitor cocktail (Boehringer). Cell lysis was achieved by sonication. The cell debris was removed by centrifugation at 10,000 g for 10 minutes. Preswollen glutathione-sepharose beads pre-equilibrated in PBS were added to the supernatant (1 ml per litre of culture). The beads were incubated with the crude protein extract for 2 hrs at 4° C. with rocking. Beads were collected and washed 3 times in PBS/0.5% Triton X-100 followed by 3 washes in PBS. Proteins were eluted from the beads by incubating in 25 mM glutathione in 50 mM Tris.Cl pH 8.0. The proteins were dialysed into a buffer containing 20 mM HEPES (pH8.0), 20% glycerol, 100 mM KCl, 0.2 mM EDTA, 1 mM dithiothreitol or PBS and stored at −80° C.

pET-30a cDNA clones were treated as above for expression except that 1 mM IPTG was used for induction and the cells were grown at 37° C. prior to induction at 30° C. The expressed protein was bound to Ni-NTA agarose beads (Qiagen) and the beads were washed with buffer containing 20 mM imidazole, 1 mM PMSF, 50 mM NaH₂PO₄, and 300 mM NaCl (pH 8.0). Recombinant protein was eluted from beads using the same buffer as above except that the concentration of imidazole was increased to 250 mM. Eluted proteins were dialysed in using the same buffer as above.

Antibody Production and Affinity Purification

Peptide antibodies to CDC5L and PLRG1 were prepared as described previously (Ajuh et al., 2000). Antibodies to bacterially expressed PLRG1 and CDC5L were also produced in sheep and rabbits (SAPU, Lanarkshire, Scotland, UK).

Splicing Assays

Nuclear extracts used in the splicing assays were obtained commercially from Computer Cell Culture Centre (Mons, Belgium). Splicing assays were done using uniformly labelled, capped pre-mRNAs incubated with nuclear extract as described previously (Lamond et al., 1997). In experiments where recombinant proteins were added to the splicing reactions, proteins that gave low yields during expression in E. coli and purification by affinity chromatography were concentrated using centrifugal filter devices (MWCO 3500) (Microcon) according to the manufacturer's instructions before addition to splicing reactions. The Adeno pre-mRNA was transcribed from Sau3AI digested plasmid pBSAd1 (Konarska and Sharp, 1987). The splicing reactions were loaded on a 10% polyacrylamide/8M urea denaturing gel and run in 1×TBE in order to separate the splicing products. When samples were to be used for the analysis of splicing complexes, the reactions were loaded onto a polyacrylamide/agarose composite gel (Konarska and Sharp, 1986) and run for about 5 hrs at 25 mA.

Immunoprecipitation of Proteins from HeLa Nuclear Extract

Immunoprecipitations of the spliceosomal proteins from nuclear extract were done using affinity purified peptide antibodies. 50 μl of nuclear extract (4-5 mg/ml) were pre-cleared for 1 hr at 4° C. on 25 μl of settled protein G or A sepharose (Pharmacia) or protein G/A agarose beads (Boehringer) that had been pre-incubated with 10 μg of sheep pre-immune IgG. The pre-cleared nuclear extract was diluted 10 times (except in immunodepletion experiments) with PBS buffer containing 0.5% Triton X-100 before adding to protein-G sepharose or agarose beads (25 μl) that had been pre-incubated with 30 pmoles of antibody for 1 hr at 4° C. Immunoprecipitations were carried out at 4° C. for 2-16 hrs. The immunoprecipitates were washed three times at 4° C. with 1 ml PBS containing 0.5% Triton X-100. Protein-G beads carrying the immune complexes were collected after each wash by centrifugation at 1500 g for 1 min. For immunodepletion experiments a higher amount of antibody (0.33 nmole/250 μg) of HeLa nuclear extract were used during the immunoprecipitation step.

SDS-PAGE and Western Blotting

SDS PAGE gel analysis was done as described previously (Laemmli, 1970). For immunoblotting, the washed immunoprecipitates were resuspended in 50 μl of 2×SDS PAGE loading buffer and heated at 95° C. for 5 mins. Approximately 10-15 μl of the supernatant were loaded on a 10% SDS PAGE gel or 4-12% pre-cast gradient gel (Novex). The separated proteins were transferred onto Hybond-C extra membrane (Amersham) by electroblotting. The membranes carrying the transferred proteins were blocked with 5% non-fat milk powder in PBS/0.3% Tween-20. The membranes were incubated with primary antibody for 1-16 hr at room temperature, washed with blocking buffer and incubated with the appropriate secondary antibody. The primary antibodies were used at the following dilutions: anti-CDC5L (1:1000); anti-PLRG1 (1:1000) and anti-SPF30 (dilution 1:2000). After washing the blots in blocking buffer 3-4 times (5 minutes per wash at room temperature), the membranes were then incubated with a secondary antibody to which has been covalently coupled horse radish peroxidase or alkaline phosphatase. Protein bands were detected by developing blots with the ECL kit (Amersham) according to the manufacturer's instructions or using NBT (60 μl of a 30 mg/ml solution)/BCIP (60 μl of a 25 mg/ml solution) in 10 ml of alkaline phosphatase buffer (0.1M NaHCO₃, 1 mM MgCl₂, pH 9.8) for colorimetric detection on the membrane.

In Vitro Transcription and Translation

The in vitro transcription and translation experiments were done with the T7 or SP6 RNA polymerase transcription/translation systems that used rabbit reticulocyte lysate (TNT systems, Promega) and L-[³⁵S] methionine (Amersham, AG1094) to produce [³⁵S] labelled proteins according to the manufacturer's recommendations. Aliquots of these reactions were used for protein-protein interaction assays as described below.

Protein Binding Assays

About 0.2 nmole of the appropriate GST-fusion recombinant protein was mixed with an equimolar amount of hexahistidine tagged bacterially expressed protein or 8-10 μl of the in vitro translation reaction above and incubated in binding buffer (250 mM NaCl, 50 mM HEPES pH 7:9, 2% BSA) at room temperature for 15 minutes. The binding reaction was then added to 25 μl of glutathione sepharose beads or antibody bound protein-G sepharose and mixed at 4° C. for 1-2 hrs. The beads were washed 3-4 times in 1 ml of PBS/0.1% Triton X-100 and resuspended in 25 μl 2×SDS PAGE loading dye before heating at 70° C. for 10 minutes or 95° C. for 5 minutes. Bound proteins were separated on a 10% or 12% SDS PAGE gel after which the gel was fixed in 50% methanol/10% acetic acid for 30 minutes. The fixed gel was then soaked in a fluorographic reagent (Amplify, Amersham) for 30 minutes before drying. Protein bands were detected by autoradiography at −80° C. after 4-16 hrs. For antibody probing, the gels containing proteins from the antibody beads were treated as described above in the SDS-PAGE and Western blotting section.

In Vitro Mutagenesis of the CDCL and PLRG1 cDNAs

Point mutations generating stop codons were inserted into the cDNAs at approximately 100 amino acids intervals by PCR using the QuikChange site directed mutagenesis kit (Stratagene) according to the manufacturer's instructions. The cDNAs were sequenced to confirm the presence of the appropriate mutations. Regions of the cDNAs involved in binding and overlapping regions were subcloned into the vectors pGEX-4T1 (Amersham-Pharmacia) or pET-30a (Novagen) for use in E. coli expression and protein-protein interaction assays.

Cell Culture and Transfection

HeLa cells were grown in Dulbecco's modified Eagles' medium supplemented with 10% foetal calf serum and 100 U/ml penicillin and streptomycin (Life Technologies Ltd). For immunofluorescence assays, cells were grown on coverslips and transfected using Effectene transfection reagent (Qiagen) according to the manufacturers instructions.

Cell Staining and Immunoflourescence Analyses

Cells were washed in PBS and fixed for 5 minutes in 3.7% (w/v) paraformaldehyde in CSK buffer (10 mM PIPES pH6.8; 10 mM NaCl; 300 mM sucrose; 3 mM MgCl₂; 2 mM EDTA) at room temperature. Permeabilisation was performed with 1% Triton X-100 in PBS for 15 minutes at room temperature. Cells were incubated with primary antibodies diluted in PBS with 1% goat serum for 35 mins to 1 hour, washed 3×10 minute with PBS, incubated for 35 mins to 1 hour with the appropriate secondary antibodies diluted in PBS with 1% goat serum and washed 3×10 minutes with PBS. Antibodies used were Y12 monoclonal antibody (anti-Sm) (Petterson et al., 1984) (dilution 1:500), rabbit anti-CDC5L (1:500) and rabbit anti-PLRG1. TRITC conjugated goat anti-mouse and Cy5 conjugated goat anti-rabbit secondary antibodies were also used (Jackson Immunochemicals). Microscopy and image analysis was carried out using a Zeiss DeltaVision Restoration microscope as described previously (Platani et al., 2000).

Peptide Synthesis

Peptides were synthesised by with N-terminal biotin by Eurogentec or Sigma-Genosys to >95% purity. Purified peptides were resuspended in water at concentrations of 2-10 mg/l and stored at −80° C.

The sequences of the designed peptides are shown in the table below.

TABLE 1

| | |
|---|---|
| Species | Peptide |

| | | |
|---|---|---|
| Homo sapiens | No. 1. | HMTTEAKRAAKMEKKMKILLGGYQ |
| | No. 2. | ELKKHEDSAIPRRLECLKEDVQRQ |
| | No. 3. | EREKELQHRYADLLLEKETLKSKF |
| | No. 4. | QYGGLLIKMKKEMKAARKAETTMH |
| | | (reverse of 1) |
| | No. 5. | EKKMKILLGGYQ |
| | No. 6. | HMTTEAKRAAKM |
| | No. 7. | KRAAKMEKKMKI |
| | No. 8 | QYGGLLIKMKKE (reverse of 5) |
| | No. 9. | KILLGGYQ |
| Drosophila melanogaster | No. 10. | EKKLKILTGGYQ |
| Lentinula (fungi) | No. 11. | EKKLGKVLGGYQ |
| Plasmodium falciparum | No. 12. | ENKYDIYTKGYQ |

Results

CDC5L and PLRG1 are co-immunodepleted from HeLa nuclear extract. The human CDC5L and PLRG1 proteins can be co-purified as part of a multi-protein complex that is essential for pre-mRNA splicing, as can their homologues in yeast (McDonald et al., 1999; Ajuh et al., 2000). It was also observed that CDC5L and PLRG1 are the most phylogenetically conserved proteins in the core CDC5L associated complex in HeLa nuclear extracts. These observations prompted the inventors to find out whether all the PLRG1 protein in HeCLa nuclear extract was co-immunoprecipitated upon immunodepletion of CDC5L. A GST-PLRG1 fusion protein was expressed in E. coli, purified and used to raise antibodies in sheep and rabbits for use in the analysis of the CDC5L-PLRG1 interaction. The immune serum obtained recognises the protein in HeLa nuclear extract as well as the bacterially expressed protein (FIG. 1B lane 1, FIG. 3A lane 3 and data not shown).

Anti-CDC5L antibodies (Ajuh et al., 2000) were used to immunodeplete CDC5L from HeLa nuclear extract and both the proteins bound on the beads and the proteins remaining in the supernatants separated by SDS PAGE and transferred onto a nitrocellulose membrane by Western blotting as described in the Materials and Methods section. Transferred proteins were probed using anti-CDC5L antibodies (FIG. 1A), anti-PLRG1 antibodies (FIG. 1B) and as a negative control, an antibody to the spliceosomal protein SPF30 (Neubauer et al., 1998), which was not identified in the human CDC5L complex (FIG. 1C). The results indicate that most of the PLRG1 is co-depleted upon immunodepletion of CDC5L from HeLa nuclear extract (FIGS. 1A and B, lanes 4 and 5). In contrast, the spliceosomal protein SPF30 remains in the supernatant i.e. is not associated with the CDC5L/PLRG1 complex. These data show that most of the PLRG1 in nuclear extract is stably associated with CDC5L.

CDC5L Co-Localises with PLRG1 In Vivo in HeLa Cell Nuclei.

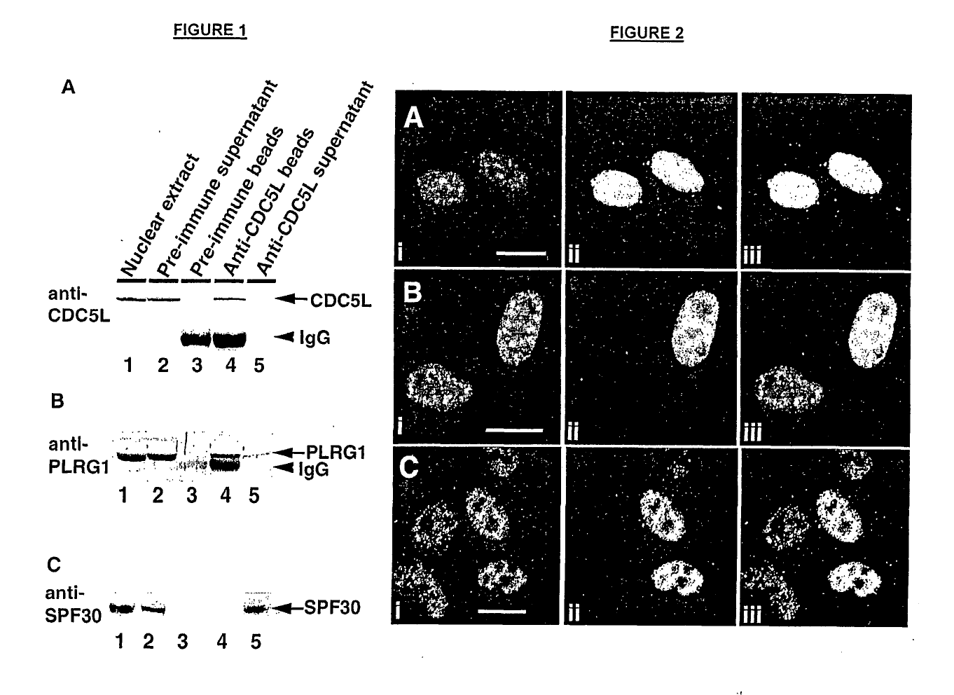

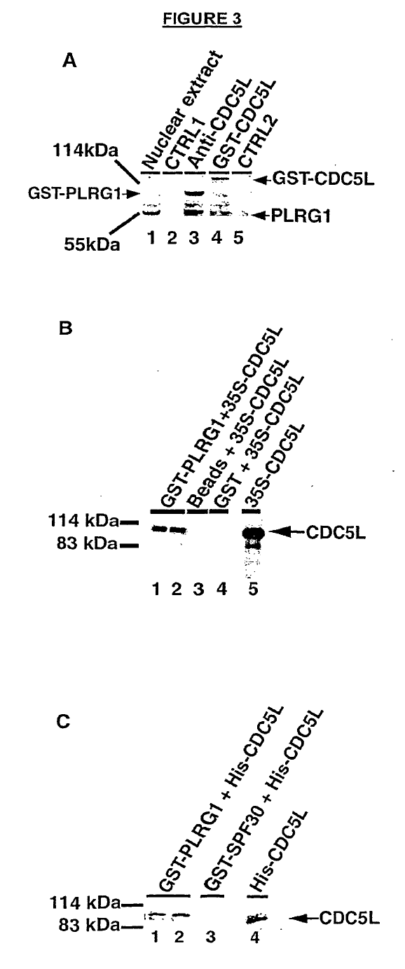

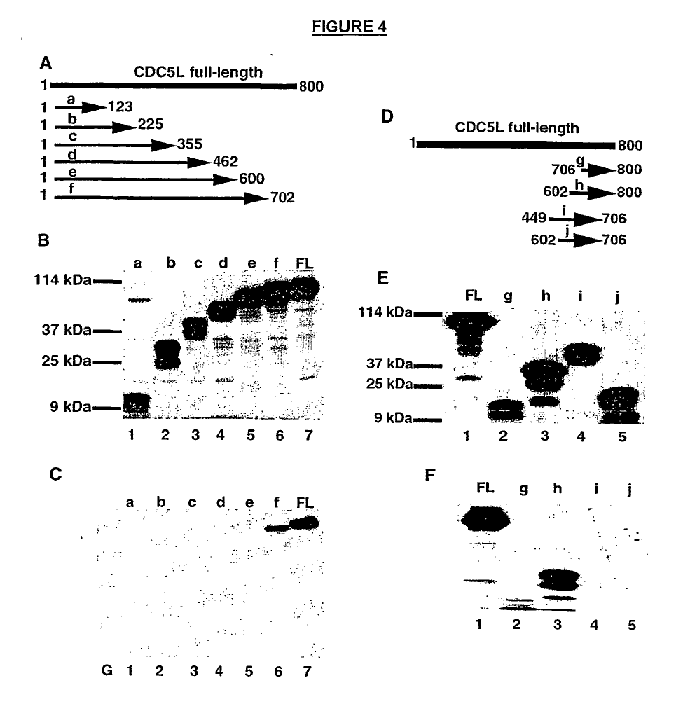

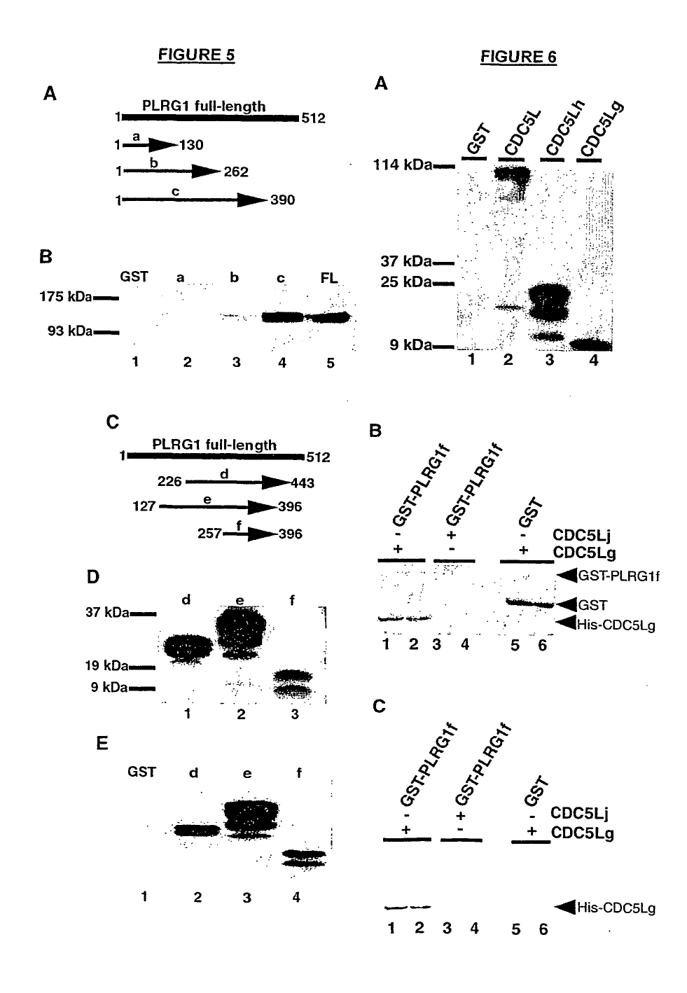

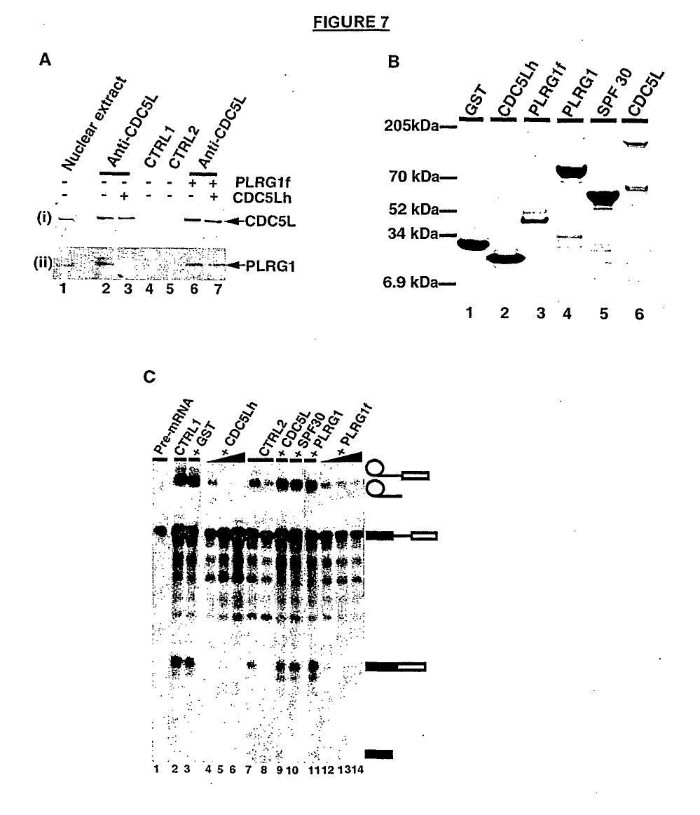

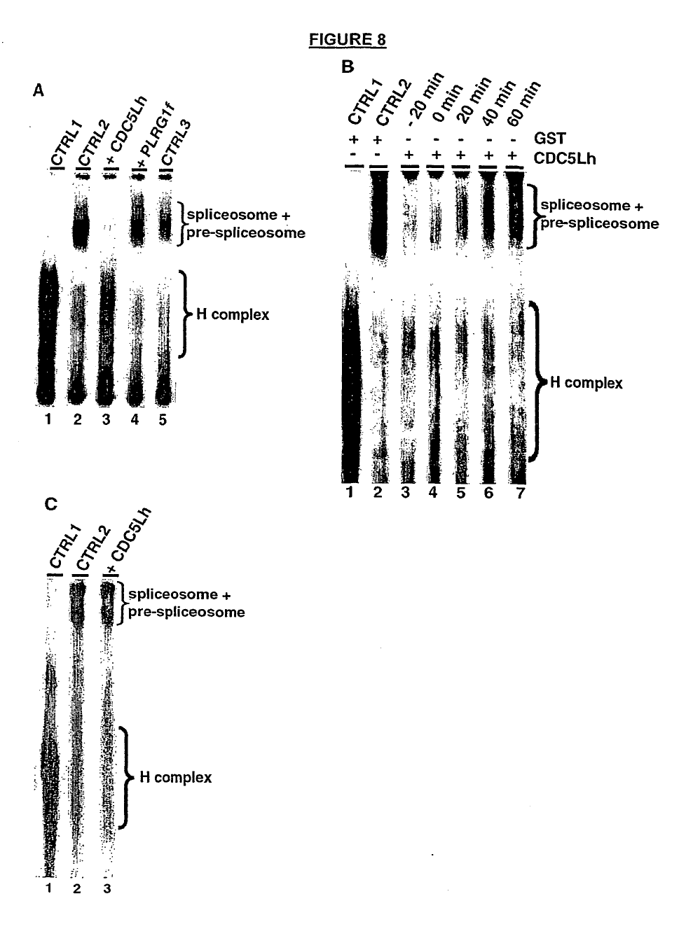

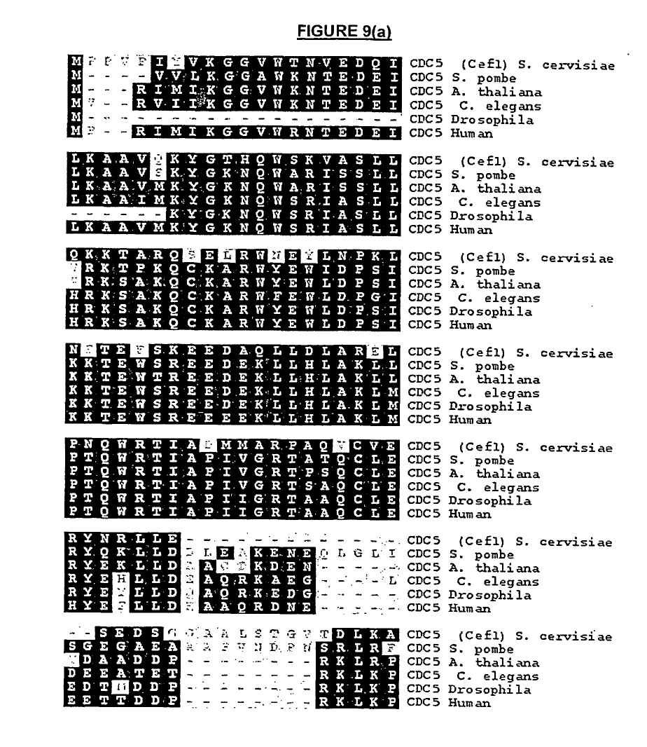

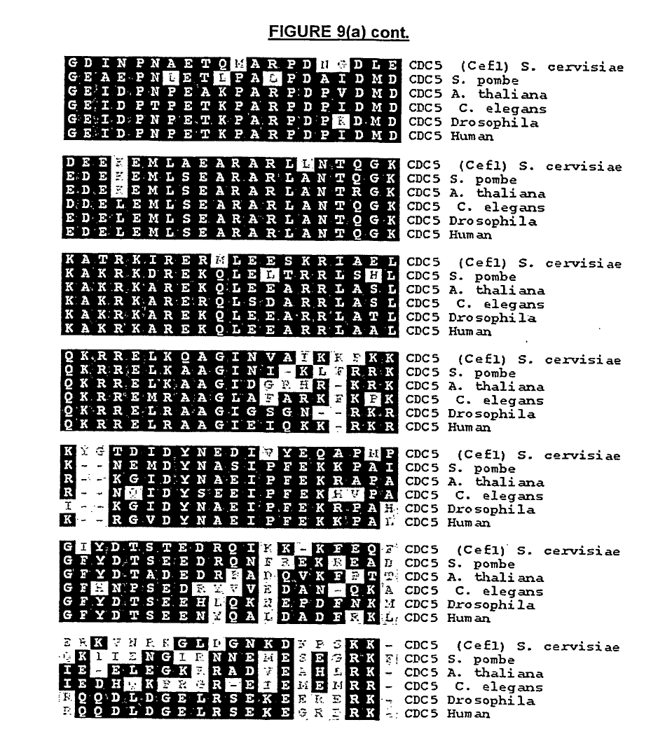

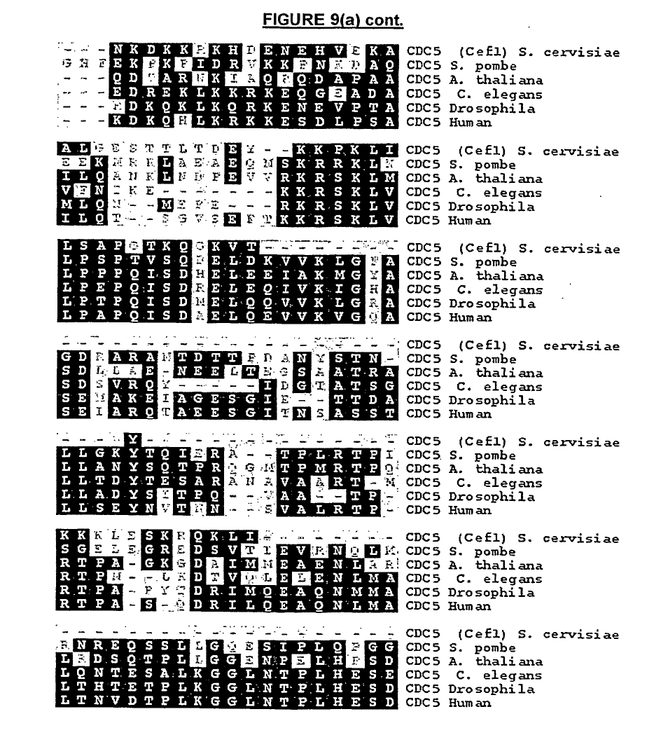

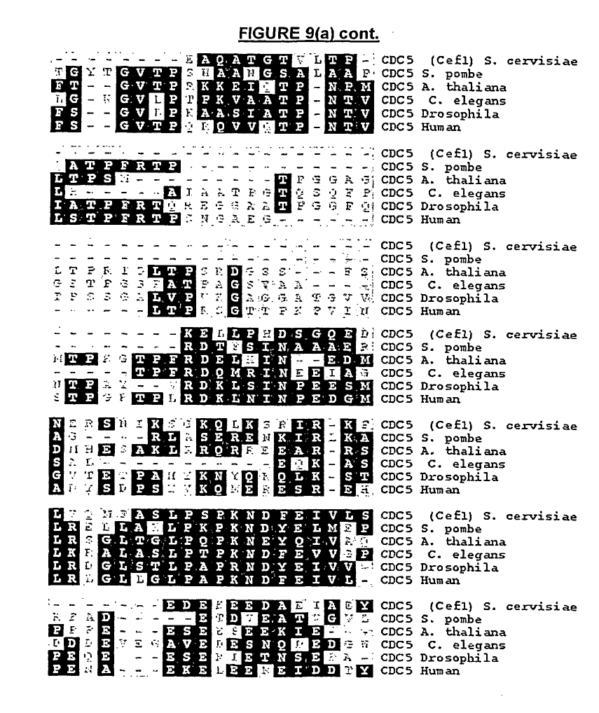

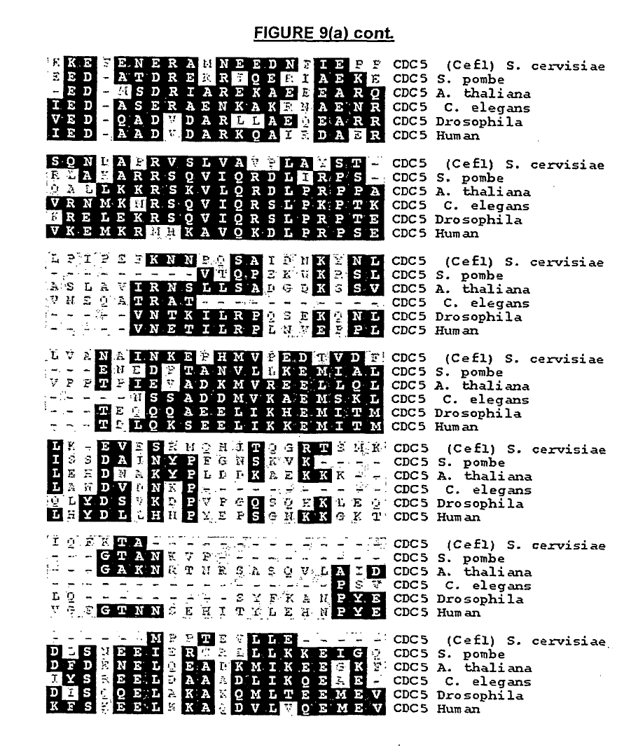

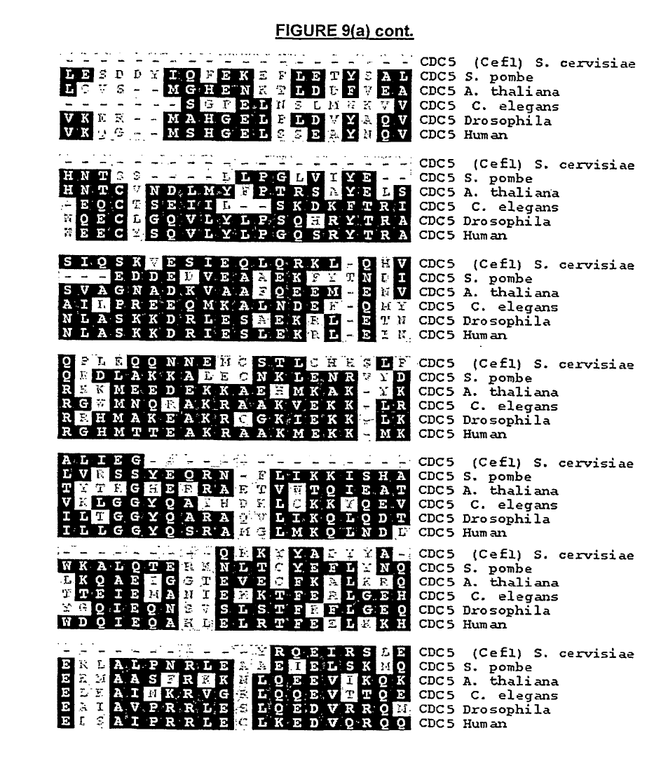

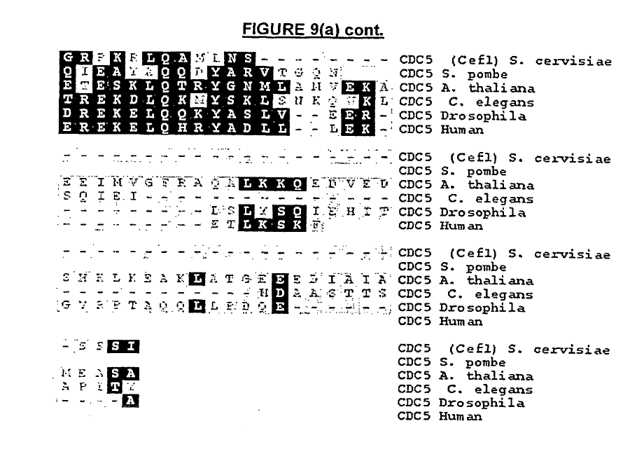

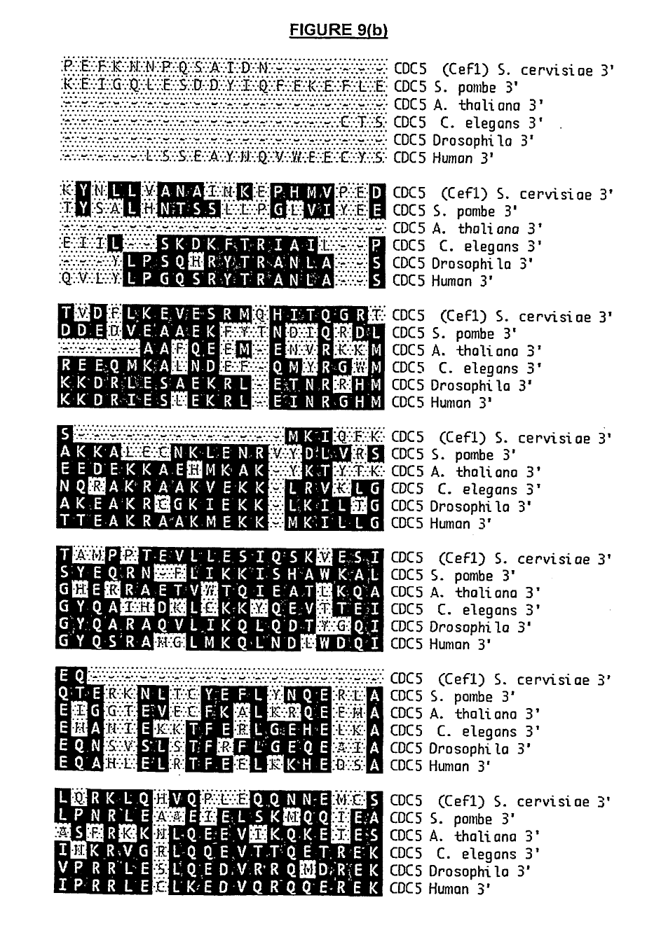

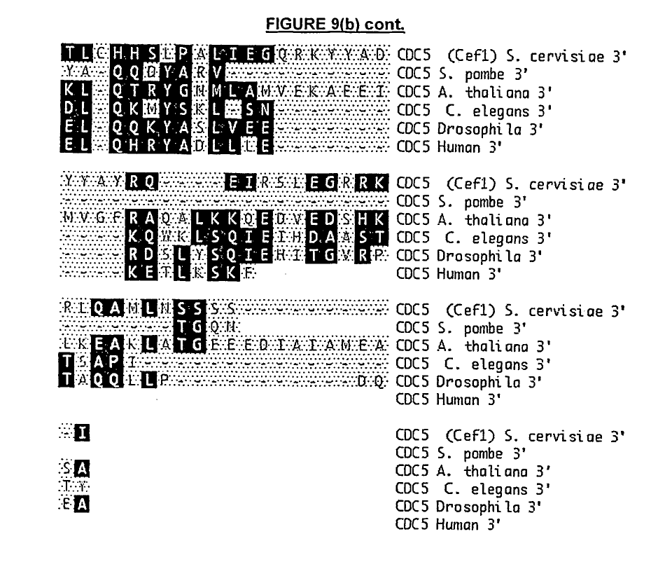

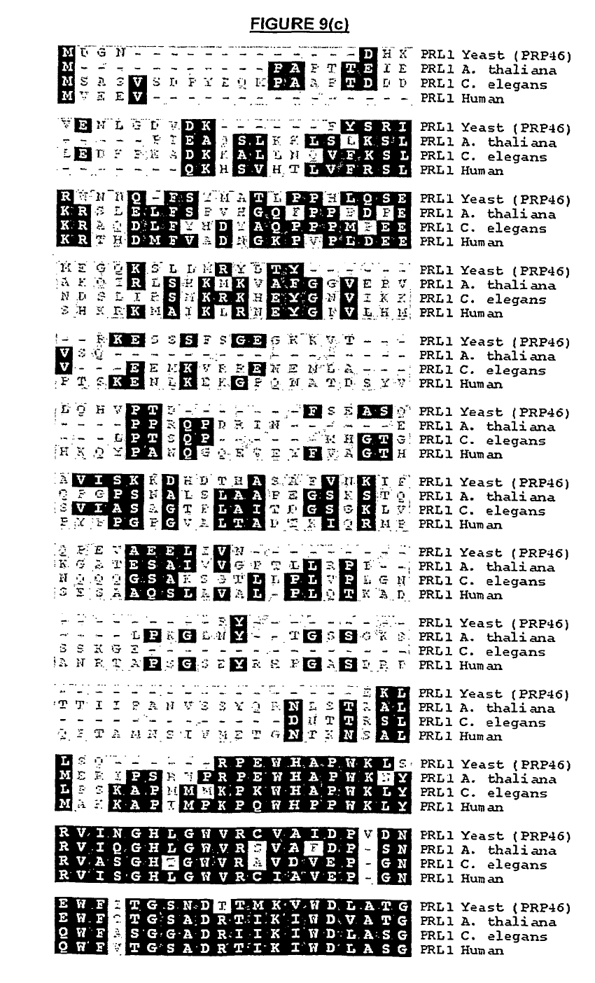

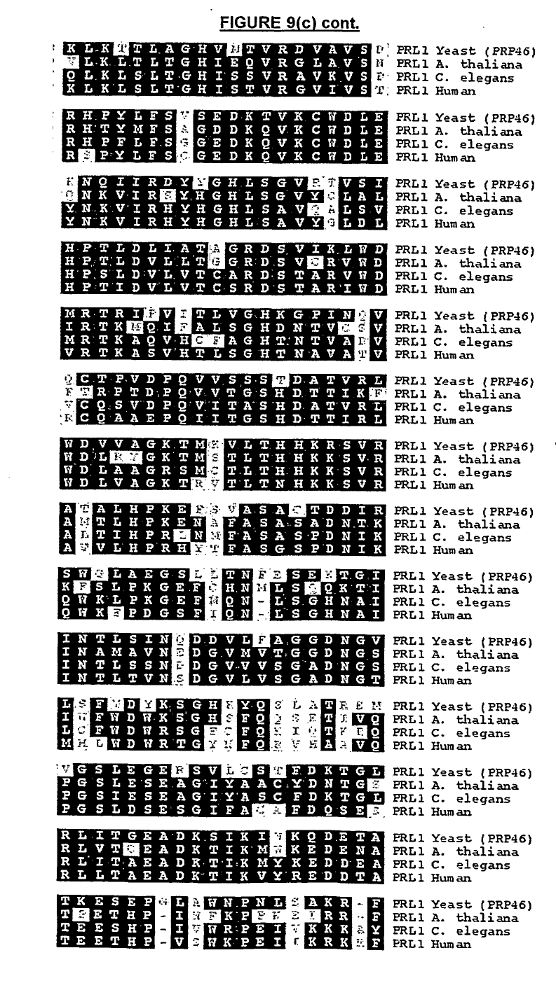

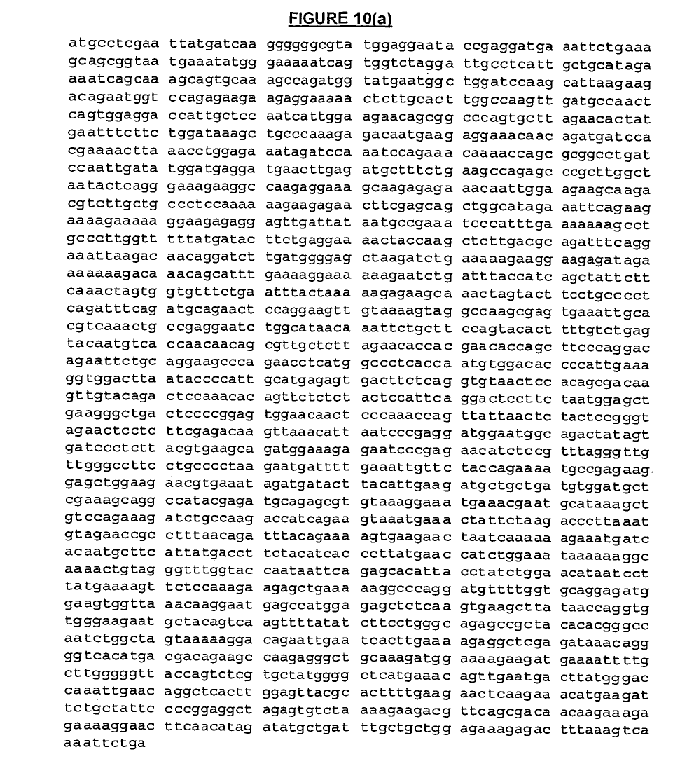

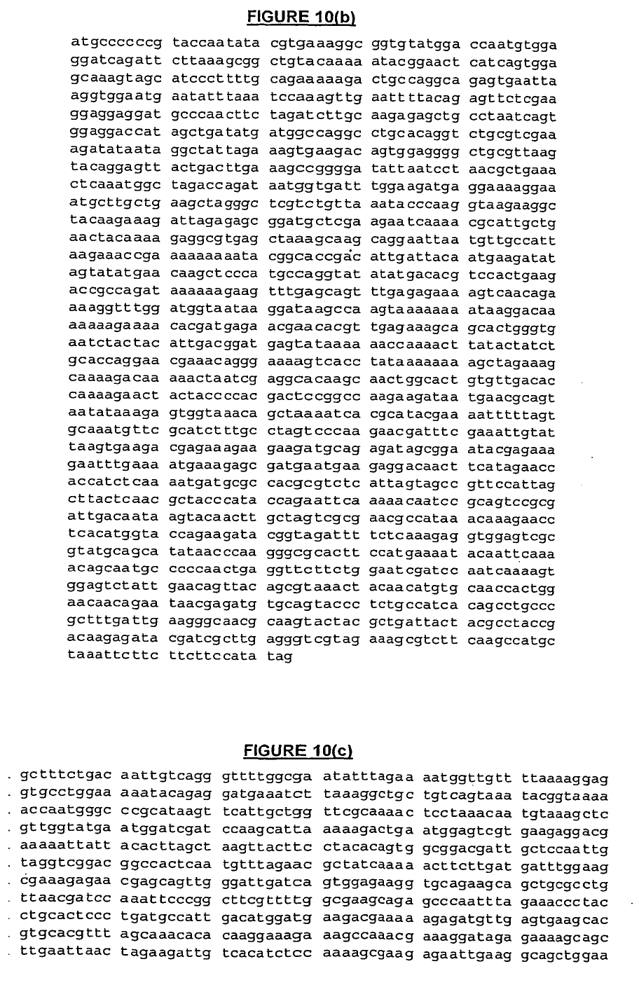

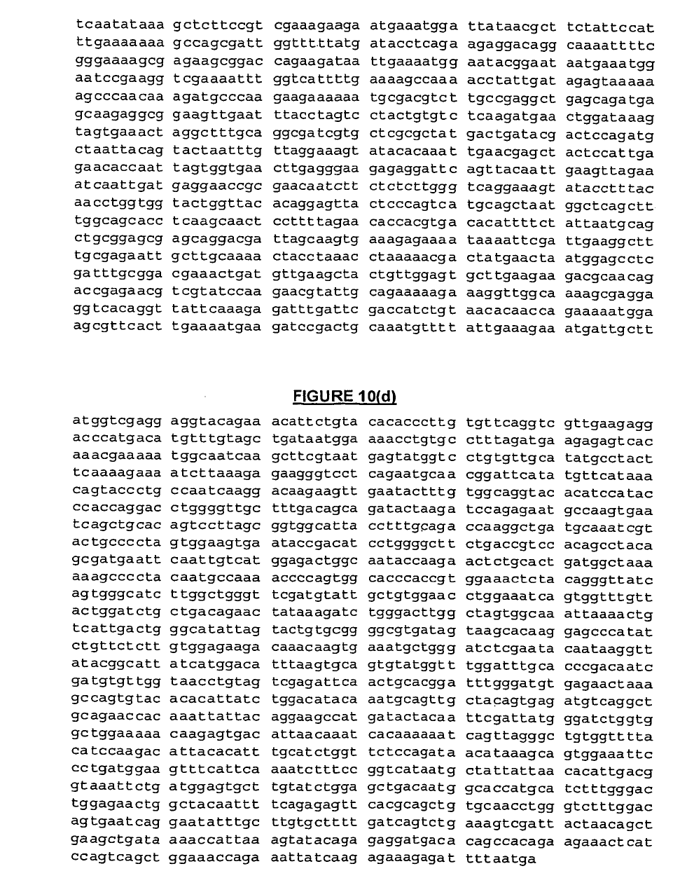

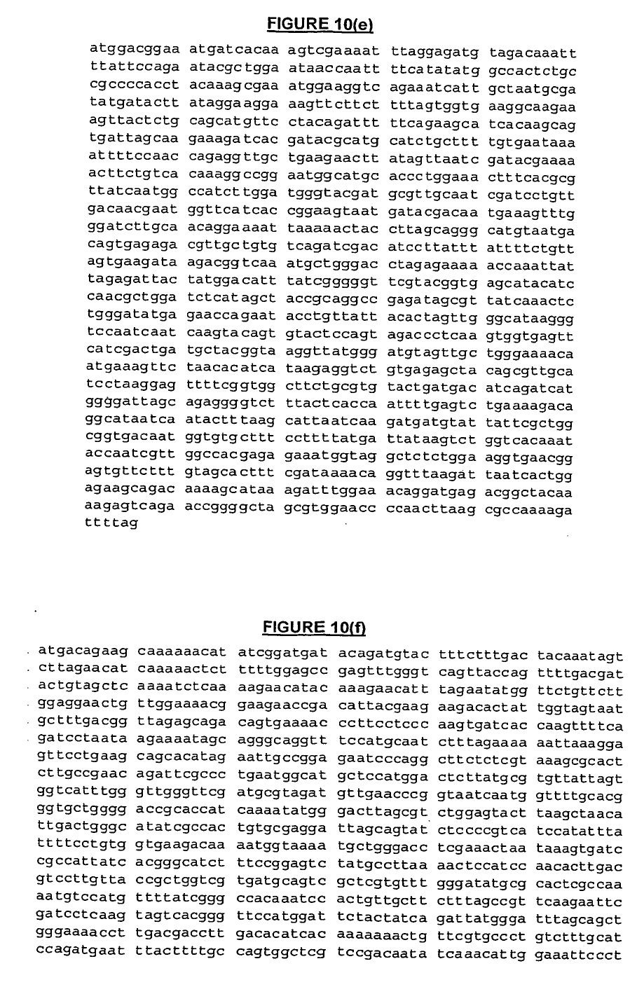

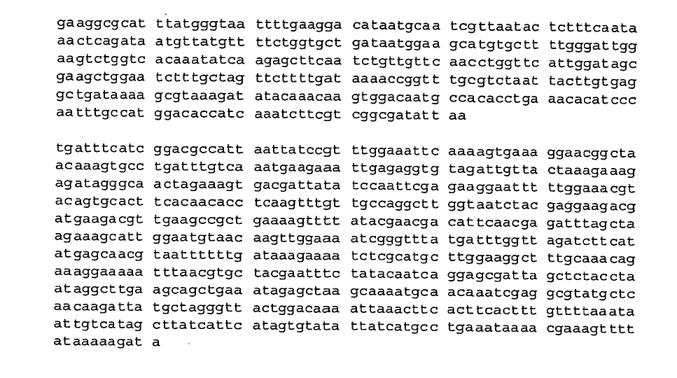

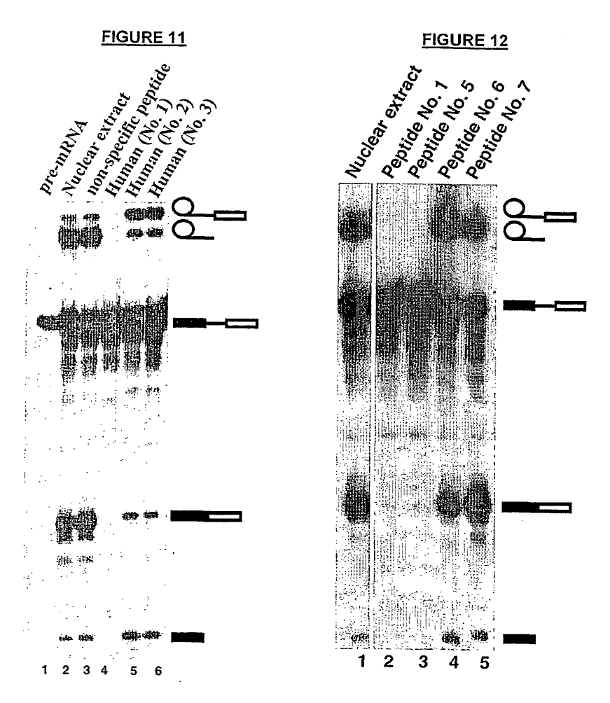

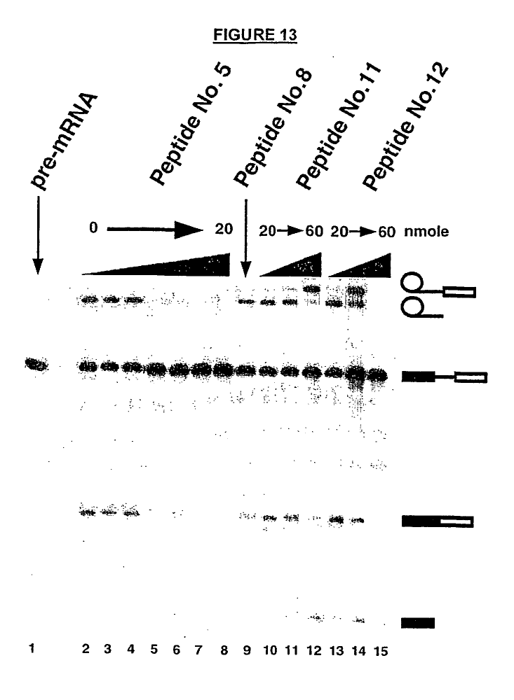

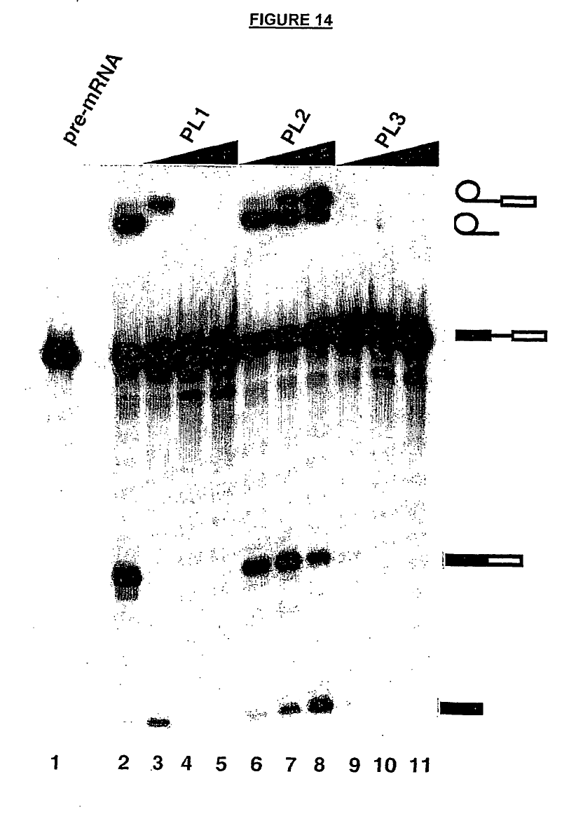

Because of the tight association between CDC5L and PLRG1 found in immunoprecipitation experiments, it was next decided to study the association of these proteins in vivo. HeLa cells were either transfected with expression vectors encoding GFP-tagged CDC5L and PLRG1 proteins, or else stained with specific antibodies. PLRG1 and CDC5L both showed a speckled nuclear staining pattern, as judged by antibody staining and by expression of GFP-fusion proteins, which co-localised with the pattern obtained by co-staining the cells with the monoclonal antibody Y12 (FIGS. 2A and B). The antibody Y12 recognises the “Sm” proteins common to each of the major splicing snRNPs (Petterson et al., 1984). When HeLa cells transiently expressing GFP-CDC5L were stained with anti-PLRG1, both proteins were found to co-localise in the same speckled structures (FIG. 2C). Similar results were obtained by staining cells transiently expressing GFP-PLRG1 with anti-CDC5L antibodies (data not shown). These results are consistent with the presence of CDC5L and PLRG1 in a common complex in vivo, but do not address whether the proteins directly interact.

CDC5L and PLRG1 Interact Directly In Vitro

The present inventors then investigated a possible direct interaction between the two proteins using in vitro methods. HeLa nuclear extracts were incubated with GST-PLRG1 and an immunoprecipitation performed on the reaction mixture using anti-CDC5L antibodies. Immunoprecipitated proteins were then separated by SDS PAGE and blotted onto nitrocellulose filters before probing with anti-PLRG1 antibodies (FIG. 3A lane 3). The results show that bacterially expressed GST-PLRG1 as well as endogenous PLRG1 are co-immunoprecipitated by anti-CDC5L antibodies and thus GST-PLRG1 will associate with CDC5L in HeLa nuclear extract. To determine whether bacterially expressed CDC5L will interact with PLRG1 in nuclear extract, the bacterially expressed protein: GST-CDC5L was incubated with HeLa nuclear extract and bound protein selected using glutathione sepharose beads. After separation by SDS PAGE and transfer of proteins to nitrocellulose membranes as described above, the blots were probed with an antibody raised against GST-PLRG1 (FIG. 3A lane 4). These results indicate that GST-CDC5L expressed in E. coli will interact with PLRG1 in HeLa nuclear extract whereas GST alone does not (FIG. 3A compare lanes 4 and 5; the PLRG1 arrow on the right of the panel shows endogenous PLRG1 pulled down by GST-CDC5L).

It is possible that the CDC5L-PLRG1 interaction may be indirect because other protein factors in the nuclear extract may be involved in mediating the interaction e.g. acting as “bridging” factors. Therefore, in vitro translated CDC5L was used in a pull-down assay with E. coli expressed GST-PLRG1 fusion protein and glutathione agarose beads (see Materials and Methods section; FIG. 3B). The results indicate that the GST-PLRG1 fusion protein will pull down L-[³⁵S] methionine-labelled in vitro translated CDC5L (FIG. 3B, lanes 1 and 2), whereas neither glutathione sepharose beads nor GST alone will (FIG. 3B, lanes 3 and 4 respectively). In order to rule out any involvement of some component in the reticulocyte lysate used for in vitro translation mediating the binding, a GST pull-down was performed using only purified, bacterially expressed proteins, i.e. GST-PLRG1 and His-tagged CDC5L (FIG. 3C). The results show that GST-PLRG1 will bind CDC5L directly in vitro, whereas GST-SPF30 (Neubauer et al., 1998), another spliceosomal protein not found in the CDC5L complex, does not interact directly with CDC5L (FIG. 3C, compare lanes 1 and 2 with lane 3). Taken together, the above data indicate that PLRG1 and CDC5L directly interact with each other.

Identification of the PLRG1 Binding Domain in CDC5L

Having shown that CDC5L and PLRG1 will interact directly in vitro, we next decided to identify the protein domain in CDC5L needed for binding to PLRG1. Protein truncation mutants were prepared by inserting stop codons at approximately 100 amino acids intervals in the CDC5L sequence progressively from the amino terminus (FIG. 4A). The mutated CDC5L expression plasmids were then used for in vitro transcription/translation to produce L-[³⁵S] methionine-labelled truncated proteins lacking overlapping regions of the carboxyl terminus of the protein (FIG. 4B). The PLRG1 interaction domain in CDC5L was determined by using full-length GST-PLRG1 in pull-down experiments with each of the respective CDC5L truncation mutants. The full-length and the ΔCDC5Lf mutant proteins were efficiently pulled down (FIG. 4C lanes 6 and 7) and very little or none of the mutants a to e were pulled down (FIG. 4C lanes 1 to 5). These results indicate that truncated proteins lacking the carboxyl terminus of the protein, as well as GST, will not bind PLRG1. This means that the PLRG1 binding region in CDC5L is located towards the carboxyl end of the protein from about amino acid residue 600 to 800.

In order to determine the binding domain for PLRG1 in the CDC5L sequence, overlapping regions towards the 3′ end of the CDC5L cDNA were subcloned (FIG. 4D) and the protein fragments translated in vitro from these cDNA regions (FIG. 4E) were used with GST-PLRG1 in pull-down experiments (FIG. 4F). The results obtained show that full-length CDC5L and the mutant proteins translated in vitro from regions g (ΔCDC5Lg) and h (ΔCDC5Lh) were pulled down using full-length GST-PLRG1 (FIG. 4F lanes 1-3 respectively). The mutant proteins obtained from the regions i (ΔCDC5Li) and j (ΔCDC5Lj) did not bind to GST-PLRG1 (FIG. 4F lanes 4 and 5). The mutant protein ΔCDC5Lg was pulled down less efficiently compared to the ΔCDC5Lh protein and full-length CDC5L (compare lanes 1 and 3 with lane 2 of FIG. 4F). These experiments show that the carboxyl terminus sequence (amino acid residues 706 to 800) is essential for binding to PLRG1 whereas sequences upstream from amino acid 706 will not bind PLRG1. However, an enhancement in binding was consistently observed (compare FIG. 4F lanes 2 and 3) when the carboxyl terminal domain of CDC5L also contained the sequences upstream from amino acid position 706 (i.e. ΔCDC5Lh). Taken together, these data indicate that although the carboxyl terminal amino acids 706-800 (ΔCDC5Lg) are essential and sufficient for PLRG1 binding, upstream amino acids can either enhance, or stabilise, this interaction.

Identification of the CDC5L Binding Domain in PLRG1

Having identified the PLRG1 binding region in CDC5L, it was next decided to determine the CDC5L binding region in PLRG1. Protein truncation mutants were engineered by inserting stop codons at regular intervals of about 130 amino acids in a 5′-3′ direction in the PLRG1 cDNA sequence (FIG. 5A). The mutations were created in the GST-PLRG1 construct and the mutant proteins expressed in E. coli and purified. The expressed proteins were then used in GST pull-downs with L-[³⁵S] methionine-labelled full-length CDC5L (FIG. 5B). The pull-down experiments showed that full-length GST-PLRG1 (FIG. 5B lane 5) and the PLRG1 truncated protein that stops at amino acid position 390 (FIG. 5B lane 4) will bind to CDC5L whereas GST alone will not (FIG. 5B lane 1). The proteins terminating at positions 130 or 262 showed little or no binding (FIG. 5B, lanes 2 and 3). In order to determine a minimum binding sequence for CDC5L in PLRG1, we next prepared sub-clones of the PLRG1 cDNA containing overlapping sequences upstream and downstream from the amino acid positions 262-390 (FIG. 5C). The cDNA sub-clones were expressed and L-[³⁵S] methionine labelled in vitro by translation in reticulocyte lysate (FIG. 5D). The labelled proteins were then used in pull-down experiments with full-length GST-CDC5L. These experiments show that the overlapping fragments containing the amino acid sequence positions 257-396 will bind to CDC5L, whereas GST alone will not interact with L-[³⁵S] methionine labelled PLRG1 (FIG. 5E compare lane 1 with lanes 2-4).

In Vitro Analysis of the Interaction between the Carboxyl Terminus Region of CDC5L and the WD Domain of PLRG1

Because all the pull-down experiments described so far involved using either full-length CDC5L to pull down PLRG1 mutants, or full-length PLRG1 to pull down CDC5L mutants, it is possible that other regions in the full-length proteins, apart from those determined using our assays, might also play a role in the binding. In order to confirm that the interacting domains in CDC5L and PLRG1 that have been identified herein are alone sufficient for binding, the respective mutant proteins containing the identified minimal binding sequences were used in pull-down experiments (FIG. 6). In the first experiment GST-ΔPLRG1f (i.e. comprising amino acid positions 257-396) was used to pull-down in vitro translated, L-[³⁵S] methionine labelled ΔCDC5Lg and ΔCDC5Lh. The results indicate that the in vitro translated carboxyl terminal sequence of CDC5L comprising amino acids 602-800 and 706-800 (FIG. 6A lanes 3 and 4) will interact with the bacterially expressed PLRG1 mutant protein containing the amino acids from position 257-396 (GST-ΔPLRG1f).

It was then decided to study the interaction between ΔPLRG1f and ΔCDC5Lg using only bacterially expressed proteins in order to exclude the possibility that some component in the reticulocyte lysate used above in the in vitro translation experiments, may mediate the interaction between the two mutant proteins. GST-ΔPLRG1f and hexahistidine-tagged ΔCDC5Lg were expressed in E. coli and affinity purified using glutathione sepharose (Pharmacia) and nickel-agarose beads (Qiagen) respectively. The purified proteins were used in a GST pull-down assay (see Materials and Methods section). Bound proteins were separated by SDS PAGE and blotted onto nitrocellulose filters. The filters were stained using Blot FastStain, (Chemicon International) according to the manufacturer's instructions, to reveal all proteins pulled down by the glutathione sepharose beads (FIG. 6B). The results show that GST-ΔPLRG1f will pull-down His-tagged ΔCDC5Lg whereas GST does not (compare FIG. 6B lanes 1 and 2 with lanes 5 and 6). It was also observed that GST-ΔPLRG1f does not bind to the truncated protein ΔCDC5Lj that lacks the carboxyl terminus sequence of CDC5L (FIG. 6B lanes 3 and 4). Since the reagent used above to stain the blot (i.e. Blot FastStain) will stain any protein transferred onto the membrane, it implies that in order to rule out the possibility that the ΔCDC5Lg band may be a partial cleavage product of GST-ΔPLRG1f, the nitrocellulose membrane was stripped and then probed with protein-S alkaline phosphatase conjugate (Novagen) according to the manufacturer's instructions (FIG. 6C). ΔCDC5Lg used in these experiments was expressed in E. coli from the plasmid pET-30a (Novagen) that contains an S-tag which is downstream from the hexahistidine sequence and upstream from the ΔCDC5Lg sequence. Protein-S specifically binds to the S-tag that is present in proteins expressed from the pET-30a plasmid vector. The results obtained after probing the membrane with protein-S alkaline phosphatase confirm the observation above that GST-ΔPLRG1f pulls down His₆-ΔCDC5Lg (FIG. 6C lanes 1 and 2) whereas GST does not (FIG. 6C lanes 5 and 6). Taken together, these results show that the truncated proteins GST-ΔPLRG1f and ΔCDC5Lg will interact directly in vitro. This means that the protein sequences that have been tested are both necessary and sufficient for binding, while other amino acid sequences outside these regions in both proteins are not strictly required for binding to occur.

Disruption of the CDC5L-PLRG1 Interaction in HeLa Nuclear Extract Inhibits Pre-mRNA Splicing.

In order to characterise further the role of these highly conserved proteins, the present inventors next investigated the effect of adding the mutant proteins containing the interaction domains in both proteins on the CDC5L-PLRG1 complex in HeLa nuclear extract (FIG. 7). It was decided to use ΔCDC5Lh for most of the subsequent experiments because this mutant protein consistently showed a higher binding efficiency to PLRG1 than ΔCDC5Lg (FIG. 4 and data not shown). ΔCDC5Lh was added to HeLa nuclear extract and wild type CDC5L immunoprecipitated from the extract using a peptide anti-CDC5L antibody (Ajuh et al., 2000), whose recognition sequence is outside the sequence contained in ΔCDC5Lh. The immunoprecipitates were then probed with anti-CDC5L antibodies (FIG. 7Ai) and anti-PLRG1 antibodies, respectively (FIG. 7Aii). The data indicate that ΔCDC5Lh will disrupt the CDC5L/PLRG1 interaction in HeLa nuclear extract because it markedly reduces co-immunoprecipitation of PLRG1 by anti-CDC5L antibodies (FIG. 7Aii lane 3). However, when the CDC5L mutant is pre-incubated with ΔPLRG1f, the disruption of the CDC5L/PLRG1 complex in HeLa nuclear extract is blocked (FIG. 7Aii lane 7). This is presumably due to the titration of the ΔCDC5Lh interaction domain by ΔPLRG1f. It was observed that although the PLRG1 mutant protein will bind to ΔCDC5Lh, it was not as efficient as ΔCDC5Lh in disrupting the CDC5L/PLRG1 interaction in HeLa nuclear extract (compare FIG. 7Aii lanes 3 and 6). The reason for this is not clear. It is possible that although ΔPLRG1f alone is sufficient for binding to CDC5L in vitro, another region of PLRG1 may be required to stabilise this interaction in HeLa nuclear extract. The absence of such a region in ΔPLRG1f may reduce the mutant protein's ability to compete with wild-type PLRG1 in nuclear extract for binding to CDC5L.

The present inventors next investigated the effect on pre-mRNA splicing of the disruption of the CDC5L-PLRG1 interaction in HeLa nuclear extract by the mutant proteins. The mutant proteins, as well as full-length proteins, were expressed in E. coli and purified as described in the Materials and Methods section. Under the expression conditions, some of the proteins gave high yields e.g. ΔCDC5Lh and PLRG1 (FIG. 7B lanes 2 and 4), while others, such as the ΔPLRG1f and CDC5L gave relatively lower yields (FIG. 7B lanes 3 and 6). When ΔCDC5Lh was added to splicing reactions, both the catalytic steps of pre-mRNA splicing were inhibited (FIG. 7C lanes 4-6) whereas splicing was unaffected upon addition of GST, GST or hexahistidine-tagged CDC5L or SPF30 (FIG. 7C lanes 3, 9 and 10 and data not shown). Addition of ΔPLRG1f alone (FIG. 7C lanes 12 to 14) inhibited splicing, although not as efficiently as ΔCDC5Lh, consistent with the previous observation of its inefficient disruption of the CDC5L/PLRG1 interaction in HeLa nuclear extract (FIG. 7A). On the other hand, when equimolar amounts of ΔCDC5Lh and ΔPLRG1f were pre-incubated together before addition to the splicing reaction, the splicing inhibition was much reduced compared to when ΔCDC5Lh is added alone (FIG. 7C, compare lanes 5 and 6 with lanes 7 and 8). This means that the interaction between the two mutant proteins has reduced their ability to disrupt the CDC5L/PLRG1 complex in the HeLa nuclear extract, thus allowing splicing to progress. The addition of full-length PLRG1 to the splicing reactions did not inhibit pre-mRNA splicing (FIG. 7C lane 11). Taken together, these results show that the interaction between CDC5L and PLRG1 in HeLa nuclear extract is important for the extract's ability to splice adeno pre-mRNA.

Effect of the Addition of ΔCDC5Lh and ΔPLRG1f Proteins to HeLa Nuclear Extract on Spliceosome Assembly.

It had been previously found that removal of CDC5L from HeLa nuclear extract by immunoprecipitation will inhibit pre-mRNA splicing, with the second catalytic step being more sensitive whereas spliceosome assembly on the pre-mRNA is not prevented (Ajuh et al., 2000). It was therefore decided to investigate whether spliceosome assembly is affected by the disruption of the CDC5L/PLRG1 interaction in HeLa nuclear extract (FIG. 8A). When ΔCDC5Lh was added to nuclear extract and the extract pre-incubated with the mutant proteins before addition to a splicing reaction, spliceosome assembly was inhibited and neither the pre-spliceosome nor spliceosome complexes were formed (FIG. 8A lane 3). The CDC5Lh mutant was found to be more efficient in this inhibition than the PLRG1f protein which showed little or no inhibition (FIG. 8A compare lanes 3 and 4). However, when equimolar amounts of the ΔPLRG1f and ΔCDC5Lh mutants were pre-incubated before addition to the nuclear extract the inhibitory effect of ΔCDC5Lh was blocked (FIG. 8A lane 5). This implies that the interaction between ΔPLRG1f and ΔCDC5Lh has prevented ΔCDC5Lh from interfering with the CDC5L-PLRG1 complex in the nuclear extract.

The present inventors next attempted to determine the step at which ΔCDC5Lh inhibits spliceosome assembly. Splicing reactions were set up as described in the Materials and Methods section and ΔCDC5Lh was added to these reactions at different time points. After the addition of ΔCDC5Lh, the reactions were allowed to proceed up to one hour. The reactions were then loaded on to native polyacrylamide/agarose composite gels and run for several hours. Bands corresponding to splicing complexes were revealed by autoradiography. The results obtained indicate that when ΔCDC5Lh was added to the nuclear extract and either pre-incubated for 20 minutes, or added immediately prior to commencement of the reaction, (time points −20 and 0 respectively, FIG. 8B lanes 3 and 4) the formation of spliceosome complexes was blocked. At the other time points i.e. adding ΔCDC5Lh between 20 and 60 minutes, progressively more spliceosomal complexes are detected assembled on the pre-mRNA (FIG. 8B lanes 5-7). These results indicate that the addition of ΔCDC5Lh inhibits any further assembly of spliceosomes after its addition to the reactions. It is also possible that the presence of the mutant protein disrupts some forms of assembled complexes.

In order to determine whether ΔCDC5Lh was disrupting assembled splicing complexes or inhibiting the formation of new complexes after its addition, a splicing reaction was set up under standard conditions and then allowed to run for about 50 minutes. The reaction was then split into two aliquots: GST was added to one aliquot i.e. the control, and to the other, an equimolar amount of ΔCDC5Lh was added. Both aliquots were then incubated for a further 10 minutes at 30° C. before being stopped. Splicing complexes formed were separated on a native gel and bands corresponding to these complexes were revealed by autoradiography (FIG. 8C). The results obtained show that ΔCDC5Lh will not disrupt spliceosomal complexes after they have been formed (FIG. 8C lane 3). Although ΔCDC5Lh was found to be unable to disrupt the spliceosome complexes, it is possible that its presence in the reaction may make the assembled complexes less stable.

Heparin is routinely added to splicing reactions at low concentrations before separation on native gels because it enhances the migration of the complexes into the gel leaving relatively less material stuck in the wells. However, heparin also has the property of disrupting aggregates and large protein complexes. It was next decided to investigate if the presence of ΔCDC5Lh caused pre-assembled spliceosome complexes on the pre-mRNA to be more sensitive to heparin treatment. Pre-mRNA splicing reactions were set up as described above and in duplicate tubes. After allowing the reactions to run for 50 minutes, GST or ΔCDC5Lh were added to separate duplicate tubes and the reaction allowed to proceed for a further 10-15 minutes. Heparin was then added to each of the reactions and the reactions incubated for 5 minutes at room temperature before loading on to native polyacrylamide/agarose gels as mentioned above. The results obtained from this experiment showed that the presence of ΔCDC5Lh does not make the spliceosomal complexes formed more sensitive to heparin treatment (data not shown). Taken together, these results indicate that ΔCDC5Lh will inhibit the formation of any new complexes after its addition to a splicing reaction and that the inhibition of spliceosome assembly occurs at an early step, perhaps prior to or, concomitant with the formation of the commitment complex

Discussion

The present inventors have recently shown that the human proteins CDC5L and PLRG1 are members of a non-snRNA containing multi-protein complex that is essential for pre-mRNA splicing in HeLa nuclear extracts (Ajuh et al., 2000). In yeast, the CDC5L homologue has also been found to be in a complex with the yeast homologue of PLRG1 (McDonald et al., 1999). However, a direct interaction between these two proteins has not previously been reported, nor has the functional significance of the association of these two highly conserved proteins in sub-spliceosomal complexes been demonstrated. Their high degree of sequence conservation across species suggests that both proteins may perform essential biological roles in the cell.

In this study it has been shown that CDC5L and PLRG1 are associated with each other in vivo and in HeLa nuclear extract. It has also been shown that CDC5L interacts directly with PLRG1 in vitro. The regions that mediate the direct interactions between the two proteins in vitro have been mapped to the carboxyl terminal domain of CDC5L and the WD40 motif region of PLRG1. By using the CDC5L mutant protein containing the PLRG1 binding domain, it has also been shown that the CDC5L/PLRG1 interaction in nuclear extract can be disrupted and that this disruption can lead to the inhibition of spliceosome assembly and pre-mRNA splicing catalysis in vitro. Taken together, the observations herein indicate that the direct interaction between the highly conserved proteins, CDC5L and PLRG1 in nuclear extract is essential for pre-mRNA splicing.

The results in this study indicate that the presence of a disrupted CDC5L complex in HeLa nuclear extract caused by the mutant protein ΔCDC5Lh interferes with the assembly of spliceosomes. This is presumably a dominant negative effect of the disrupted complex because its presence is not strictly required for assembly. This possibility is supported by the observations that the mutant protein ΔPLRG1f, which is not efficient in disrupting the CDC5L-PLRG1 interaction in HeLa nuclear extracts, does not prevent spliceosome assembly. Secondly, the fact that ΔCDC5Lh blocks formation of splicing complexes once added to a reaction but does not disrupt complexes that have already been formed, further supports the view that the presence of a disrupted CDC5L complex may interfere with the de novo assembly of spliceosomal complexes. Therefore, while the present inventors cannot rigorously exclude the possibility that the previous observations that immunodepletion of CDC5L from HeLa nuclear extract inhibits catalysis but not spliceosome assembly (Ajuh et al., 2000) may be due to incomplete removal of the interacting proteins with the low level of remaining proteins sufficient to promote spliceosome assembly and, to a lesser extent, the first catalytic step of splicing, the alternative explanation is favoured. Namely that the disrupted components of the CDC5L complex inhibit spliceosome formation, although the complex is not strictly required for assembly to proceed.

Although the CDC5L and PLRG1 proteins and their homologues have previously been identified in spliceosomal protein complexes (Neubauer et al., 1998; Tsai et al., 1999; McDonald et al., 1999; Ajuh et al, 2000) their possible direct interactions with other splicing factors have not yet been investigated in detail. Interestingly, in yeast, the CDC5L homologue CEF1 has been shown to interact with the splicing factor Prp19p through a region in the carboxyl terminal half of the protein and this region has also been shown to be essential for CEF1 self-interaction (Tsai et al., 1999). Saccharomyces cerevisiae Prp19p is required for the first catalytic step of splicing and may associate with the spliceosome either after, or simultaneously, with U4 snRNP dissociation from the complex. It has therefore been suggested that Prp19p may play a role in mediating the structural rearrangements in the spliceosome during U4 dissociation (Tam et al., 1993; Chen et al., 1998). The human homologue of yeast Prp19 has not yet been characterised, although we have identified a Prp19-like protein in our recent study of the CDC5L complex in HeLa nuclear extract (Ajuh et al., 2000). It is thus possible that both Prp19 and PLRG1 may interact, either simultaneously, or alternatively, with the carboxyl terminal domain of CDC5L. The length of the Prp19 binding domain identified in the yeast CEF1 protein is about 30 amino acids. The equivalent sequence to the above region in humans, determined by comparative sequence analysis, is about 100 amino acids upstream from the 94 amino acid region we have identified as the PLRG1 binding sequence in CDC5L. The involvement of the WD motif domain of PLRG1 in binding to CDC5L is consistent with the suggestion that proteins containing these regions may have a role in multiple simultaneous or consecutive protein-protein interactions (Smith et al., 1999). Although PLRG1 has not previously been shown to interact directly with specific pre-mRNA splicing factors, there is some evidence that this protein may interact with other cellular proteins not directly involved in splicing, such as protein kinase C-βII and α-importin ATHKAP2 (Nemeth et al., 1998).

In yeast, the CDC5L homologue CEF1 has been shown to mediate the targeting of the CDC5L associated complex to the spliceosome as a single multi-protein complex (Tsai et al., 1999). These findings are consistent with the observations that ΔPLRG1f on its own did not significantly inhibit spliceosome assembly, whereas ΔCDC5Lh was more efficient in inhibiting assembly. This may be because the CDC5L mutant protein containing the PLRG1 binding domain may not only disrupt the CDC5L/PLRG1 complex in nuclear extract but also interfere with interactions between other splicing factors that need to be targeted to the assembling spliceosome. Secondly, because the carboxyl terminal region has been shown to be needed for self-interaction in the S. cerevisiae homologue of CDC5L (CEF1) (Tsai et al., 1999), it is possible that our CDC5L mutant inhibits CDC5L self-interactions. These self-interactions may be essential for the function of the protein in pre-mRNA splicing. Further studies on characterising the direct protein-protein interactions between core members of the CDC5L, associated complex should shed more light on how these interactions affect the pre-mRNA splicing mechanism.

CDC5L and its homologues in other species have already been implicated in several cellular roles in addition to pre-mRNA splicing; for example, cell cycle progression (Nasmyth and Nurse, 1981; Ohi et al., 1994; Bernstein and Coughlin, 1998), sequence specific double stranded DNA binding and transcription activation in HeLa cells. The transcription activation ability of the protein was determined by using a vector containing a luciferase reporter fused downstream from a CDC5L binding site. It was observed that luciferase activity is increased by about 28 fold when the HeLa cells were co-transfected with a plasmid expressing CDC5L (Lei et al., 2000). It is thus possible that CDC5L may be needed in the cell for pre-mRNA splicing, transcription and cell division. Like CDC5L, the PLRG1 homologue in Arabidopsis thaliana called PRL1 has been shown to have a pleiotropic role in several regulatory pathways in this species. These include glucose metabolism, hormonal responses, transcription of genes regulated by sucrose and cytokinin, and regulation of SNF1-like kinases (Nemeth et al., 1998; Bhalerao et al., 1999). Thus, the CDC5L and PLRG1 proteins in eukaryotes may be directly involved in several cellular functions as mentioned above. Alternatively, it may be that some of the above observations are an indirect consequence of changes in the expression of specific genes in these organisms mediated by the role of CDC5L and PLRG1 in the mechanism of pre-mRNA splicing. Further studies are now required to characterise in more detail the interaction of the CDC5L/PLRG1 complex with other splicing factors in order to understand the contribution of such interactions to the splicing reaction.

Use of Peptides Derived from the Binding Regions of CDC5L; and PLRG1 in Inhibiting Pre-mRNA Splicing

A 24-Mer Peptide from the CDC5L Region that Binds PLRG1 Will Inhibit Splicing

24 mer peptides were designed as shown in table 1 for Homo sapiens (Nos. 1-3) and 16 nmol of purified peptides were added to 30 μL splicing reactions (FIG. 11). Splicing intermediates were then separated on a denaturing gel as described in the Materials and Methods section. The results obtained indicate that the human peptide No. 1 will inhibit splicing (FIG. 11 Lane 3) whereas a non-specific peptide or the other human peptides (Lanes 5 and 6) did not inhibit splicing, Down the right hand side of the Figure and FIGS. 12, 13 and 14 there is a schematic representation of products observed on the gels. The starting product (a) consists of exon 1 and exon 2 together with intervening intro, this is the pre-mRNA substrate. In step 1 the free 5′ exon 1 intermediate (b) and the lariat intro and exon 2 intermediate (c) are generated. Further splicing produces the spliced exons 1 and 2 (d) and the excised lariat product (e). This is further explained in detail in Biochimca et Biophysica Acta (1993) 1173, 247-265 (G. M. Lamm and A. I. Lamond).

A 12 Mer Peptide Designed from Human Peptide No. 1 Will Also Inhibit Splicing

Overlapping peptide sequences were designed from human peptide sequence No. 1 (ie. numbers 5-7). The peptides were synthesised and purified by Sigma-Genosys to >95% purity. The purified peptides were added to splicing reactions as above. The results obtained show that peptides number 1 and 5 will inhibit splicing (FIG. 12, Lanes 2 and 3) whereas the other peptides do no. Peptide number 8 (the reverse sequence to peptide No. 5) was used as a control.

Species Specific and Dose Dependent Inhibition of Pre-mRNA Splicing by CDCL Related Peptides

Splicing assays were set up as described above using the peptides number 5, 10, 11 and 12. Peptides were added to the splicing reactions in amounts ranging from 3 to 60 nmole (FIG. 13). The results from this experiment indicate that 15-20 nmole of the human peptide will inhibit splicing whereas splicing inhibition only starts at 60 nmole when using the fungal peptide (FIG. 13, Lane 12). The Plasmodium peptide starts inhibiting at about 40 nmole (FIG. 13, Lane 14) and completely inhibits splicing at 60 nmole (FIG. 13, Lane 15). The reverse peptides were not able to inhibit splicing (FIG. 13, Lane 14 and data not shown). The Drosophila peptide did not inhibit splicing at 20 nmole. However, higher concentrations of this peptide have not yet been tested. Based on the above date, our prediction is that this peptide would also inhibit splicing when used at levels over 20-60 nmole.

Inhibition of Pre-mRNA Splicing by PLRG1 Derived Peptides

Splicing assays were set up as described above using peptides PL1, PL2 and PL3. Peptides were added to the splicing reactions ranging from 7 to 21 nmoles (FIG. 14). As can be seen PL1 and PL3 have the ability to inhibit pre-mRNA splicing.

### References

Ajuh, P. Kuster B., Panov, K., Zomerdijk, J. C. B. M., Mann M., and Lamond, A. I. (2000) Functional analysis of the human CDC5L complex and identification of its components by mass spectrometry. EMBO J. 19, 6569-6581.

Bernstein, H. S. and Coughlin, S. R. (1997) A putative human transcription factor implicated in mitogen-activated signaling. J. Biol. Chem. 272: 5833-5837.

Bernstein, H. S. and Coughlin, S. R. (1998) A mammalian homolog of fission yeast CDC5 regulates G2 progression and mitotic entry. J. Biol. Chem. 273: 4666-4671.

Bhalerao, R. P., Salchert, K., Bako, L., Okresz, L., Szabados, L., Muranaka, T., Machida, Y., Schell, J. and Koncz, C. (1999) Regulatory interaction of PRL1 WD protein with Arabidopsis SNF1-like protein kinases. Proc Natl Acad Sci U S A. 96: 5322-7.

Burns, C. G., Ohi, R., Krainer, A. R., Gould, K. L. (1999) Evidence that Myb-related CDC5 proteins are required for pre-mRNA splicing. Proc. Natl. Acad. Sci. USA. 96, 13789-13794.

Chen, H. R., Jan, S. P., Tsao, T. Y., Sheu, Y.J., Banroques, J., Cheng, S. C. (1998) Snt309p, a component of the Prp19p-associated complex that interacts with Prp19p and associates with the spliceosome simultaneously with or immediately after dissociation of U4 in the same manner as Prp19p. Mol. Cell. Biol. 18: 2196-2204

Green, S., I. Issemann, and Sheer, E. (1988) A versatile in vivo and in vitro eukaryotic expression vector for protein engineering. Nucleic Acids Res. 16: 369.

Hirayama, T. and Shinozaki, K. (1996). A cdc5+ homolog of a higher plant, Arabidopsis thaliana. Proc. Natl. Acad. USA. 93: 13371-13376.

Janda, L., P. Tichy, J. Spizek, and M. Petricek. (1996) A deduced Thermonospora curvata protein containing serine/threonine protein kinase and WD-repeat domains. J. Bacteriol. 178: 1487-1489.

Konarska, M. M. and Sharp, P. A. (1986) Electrophoretic separation of complexes involved in the splicing of precursors to mRNAs. Cell 46, 845-55

Konarska, M. M. and Sharp, P. A. (1987) Interactions between small nuclear ribonucleoprotein particles in formation of spliceosomes. Cell 49, 763-774.

Krämer, A. (1995) The biochemistry of pre-mRNA splicing. In Lamond, A. I. (ed.) Pre-mRNA processing. Springer-Verlag, Heidelberg, Germany, pp. 39-64.

Krämer, A. (1996) The structure and function of proteins in mammalian pre-mRNA splicing. Annu. Rev. Biochem., 65, 357-409.

Laemmli, U.K. (1970) Cleavage of structural proteins during the assembly of the head of bacteriophage T4. Nature, 227, 680-685

Lamond A. I., Konarska, M. M. and Sharp, P. A. (1987) A mutational analysis of spliceosome assembly: evidence for splice site collaboration during spliceosome formation. Genes and Develop., 1, 532-543.

Lei, X. H., Shen, X., Xu, X. Q., and Bernstein, H. S. (2000) Human Cdc5, a regulator of mitotic entry, can act as a site-specific DNA binding protein. J Cell Sci. 113: 4523-4531.

McDonald, W. H., Ohi, R., Smelkova, N., Frendewey, D. and Gould, K. L. (1999) Myb-related fission yeast cdc5p is a component of a 40S snRNP-containing complex and is essential for pre-mRNA splicing. Mol. Cell. Biol., 19, 5352-5362.

Nasmyth, K. and Nurse, P. (1981) Cell division cycle mutants altered in DNA replication and mitosis in the fission yeast Schizosaccharomyces pombe. Mol Gen Genet, 182:119-24

Neer E. J., Schmidt, C. J., Nambudripad, R, and Smith, T. F. (1994) The ancient regulatory protein family of WD-repeat domains-Progress. Nature 371: 297-300.

Nemeth, K., K. Salchert, P. Putnoky, R. Bhalerao, Z. Konc-Kalman, B. Stankovic-Stangeland, L. Bako, J. Mathur, L., Okresz, S. Stabel, P. Geigenberger, M. Stitt, G. P. Redei, J. Schell, and C. Koncz. (1998) Pleiotropic control of glucose and hormone responses by PRL1, a nuclear WD protein, in Arabidopsis. Genes & Dev. 12: 3059-3073.

Neubauer, G., King, A., Rappsilber, J., Calvio, C., Watson, M., Ajuh, P., Sleeman, J., Lamond, A. and Mann, M. (1998) Mass spectrometry and EST-database searching allows characterisation of the multi-protein spliceosome complex. Nat. Genet., 20, 46-50.

Ohi, R., Feoktistova, A., McCann, S., Valantine, V., Look, A. T., Lipsick, J. S., and. Gould, K. L (1998) Myb-related Schizosacharomyces pombe cdc5p is structurally and functionally conserved in eukaryotes. Mol. Cell. Biol. 18: 4097-4108.

Ohi, R., McCollum, D., Hirani, B., Den Haese, G. J., Zhang, X., Burke, J. D., Turner, K. and Gould, K. L. (1994) The Schizosacharomyces pombe cdc5+ gene encodes an essential protein with homology to c-Myb. EMBO J. 13: 471-483.

Petterson, I., Hinterberger, M., Mimori, T., Gottlieb, E. and Steitz, J. A., (1984) The structure of mammalian small nuclear ribonucleoproteins. Identification of multiple protein components reactive with anti-(U1) ribonucleoprotein and anti-Sm autoantibodies. J Biol Chem 259: 5907-5914

Platani, M., Goldberg, I., Swedlow, J. R., and Lamond A. I. (2000) In Vivo Analysis of Cajal Body Movement, Separation, and Joining in Live Human Cells. J. Cell Biol. 151: 1561-1574.

Potashkin, J., Kim, D., Fons, M., Humphrey, T. and Frendewey, D. (1998) Cell-division-cycle defects associated with fission yeast pre-mRNA splicing mutants. Curr Genet. 34: 153-63.

Reed, R. and Palandjian, L. (1997) Spliceosome assembly. In Krainer, A. R. (ed.) Eukatyotic mRNA processing. Oxford University Press, New York, N.Y. pp. 130-173.

Smith, T. F., Gaitatzes, C., Saxena, K. and Neer, E. J. (1999) The WD repeat: a common architecture for diverse functions. Trends Biochem Sci., 24, 181-185.

Staley, J. P. and Guthrie C. (1998) Mechanical devices of the spliceosome: motors, clocks, springs, and things. Cell. 92, 15-26.

Stukenberg, P. T., Lustig, K. D., McGarry, T. J., King, R. W., Kuang J. and. Kirschner, M. W (1997) Systematic identification of mitotic phosphoproteins. Curr. Biol. 7: 338-348.

Tam, W. Y., Lee, K. R. and Cheng, S. C. (1993) Yeast precursor mRNA processing protein PRP19 associates with the spliceosome concomitant with or just after dissociation of U4 small nuclear RNA. Proc Natl Acad Sci U S A. 90: 10821-10825.

Tsai, W. Y., Chow, Y. T., Chen, H. R., Huang, K. T., Hong, R. I., Jan, S. P., Kuo, N. Y., Tsao, T. Y., Chen C. H. and Cheng, S. C. (1999) Cef1p is a component of the Prp19p-associated complex and essential for pre-mRNA splicing. J Biol. Chem., 274, 9455-9462.

Will, C. L. and Lurhmann, R. (1997) snRNP structure and function. In Eukaryotic mRNA processing (ed. A. R. Krainer), pp. 130-173. Oxford University Press, New York, N.Y.

Xia, G., Ramachandran, S., Hong, Y., Chan, Y. S., Simanis, V. and Chua, N. H. (1996) Identification of plant cytoskeletal, cell cycle-related and polarity-related proteins using Schizosacharomyces pombe. Plant J. 10: 761-769.

1. A method for identifying a substance capable of modulating an interaction between (i) a CDC5L polypeptide or a homologue thereof, or a derivative thereof, and (ii) a PLRG1 polypeptide, or a homologue thereof, or a derivative thereof, which method comprises:
a) providing a CDC5L polypeptide or a homologue, or a derivative thereof, as a first component and a PLRG1 polypeptide or a homologue, or a derivative thereof, as a second component;
b) contacting the two components with a test substance under conditions that would permit the two components to interact in the absence of said test substance; and
c) determining whether said substance modulates the interaction between the first and second components.

2. The method according to claim 1 further comprising:
d) administering a substance which has been determined to disrupt the interaction between the first and second components to a eukaryotic cell; and
e) determining the effect of the substance on the cell.

3. The method according to claim 1, wherein the CDC5L polypeptide comprises amino acid residues 602-800 of the human sequence or corresponding region from a species specific homologue.

4. The method according to claim 1, wherein the PLRG1 polypeptide comprises amino acid residues 257-396 of the human sequence or a corresponding region from a species specific homologue.

5. The method according to claim 1, wherein the CDC5L and/or PLR1 polypeptides are obtained from mammalian, yeast or fungal sources.

6. The method according to claim 1, wherein said CDC5L and/or PLRG1 polypeptides are recombinantly produced.

7. A substance capable of modulating an interaction between (i) a CDC5L polypeptide or a homologue thereof, or a derivative thereof, and (ii) PLRG1 or a homologue thereof, or a derivative thereof, identified by a method according to claim 1, for use in treating the human or animal body by therapy or for use in diagnosis.

8. Use of a substance according to claim 7 for the preparation of a medicament for the prevention or treatment of cancer, or fungal infections.

9. A substance capable of modulating an interaction between (i) a CDC5L polypeptide or a homologue thereof, or a derivative thereof, and (ii) PLRG1 or a homologue thereof, or a derivatives thereof, identified by a method according to claim 1 for use in regulating mRNA splicing.

10. Use of the substance according to claim 9 for the manufacture of a medicament for inhibiting protein synthesis by disrupting mRNA splicing.

11. A method of regulating and/or disrupting the cell cycle in a eukaryotic cell, which method comprises administering to said cell a substance capable of modulating an interaction between (i) a CDC5L polypeptide or a homologue thereof, or a derivative thereof, and (ii) PLRG1 or a homologue thereof, or a derivative thereof.

12. A peptide comprising the sequence:

| | |
|---|---|
| a) | EKKMKILLGGYQ; |
| b) | EKKLKILTGGYZ; |
| c) | EKKLGKVLGGYD; |
| d) | ENKYDIYTKGYQ; |
| e) | PYLFSCCEDKQVKCWDLEYNKVIRHYHGHL; |
| or | |
| f) | PQIITGSHDTTIRLWDLVAGKTRVTLTHNK |

for use in inhibiting pre-mRNA splicing.

13. Use of a peptide according to claim 12 in therapy or diagnosis.

14. Use of a peptide:

| | |
|---|---|
| a) | EKKMKILLGGYQ; |
| b) | PYLFSCCEDKQVKCWDLEYNKVIRHYHGHL; |
| or | |
| c) | PQIITGSHDTTIRLWDLVAGKTRVTLTNHK |

for the manufacture of a medicament for treating diseases associated with undesirable pre-mRNA splicing.

15. The use according to claim 14 wherein said diseases are cancer or psoriasis.

16. Use of the peptide EKKLKILTGGYZ for the manufacture of a pesticide.

17. The use according to claim 16 for attacking Drosophila melanogaster.

18. Use of the peptide EKKLGKVLGGYD for the manufacture of a medicament for treating a fungal infection.

19. Use of the peptide ENKYDIYTKKGYQ for the manufacture of a medicament for treating a yeast infection.

20. A peptide comprising the sequence EKKMKILLGGYQ from positions 714-725 of the human CDC5L peptide sequence or corresponding sequence from a species specific homologue for use in inhibiting pre-mRNA splicing.

21. Use of the peptide according to claim 20 in therapy or diagnosis.

22. A peptide comprising the sequence:

| | |
|---|---|
| [[e]]a) | |
| PYLFSCCEDKQVKCWDLEYNKVIRHYHGHL | (residues 259-288); |
| or | |
| [[f]]b) | |
| PQIITGSHDTTIRLWDLVAGKTRVTLTNHK | (residues 343-372) |

from the human PLRG1 peptide sequence or corresponding sequence from a species specific homologue for use in inhibiting pre-mRNA splicing.

23. Use of the peptide according to claim 22 in therapy or diagnosis.

24. A pharmaceutical formulation comprising the peptide according to claim 12 together with a pharmaceutical acceptable excipient.

25. A pharmaceutical formulation comprising the peptide according to claim 20 together with a pharmaceutical acceptable excipient.

26. A pharmaceutical formulation comprising the peptide according to claim 22 together with a pharmaceutical acceptable excipient.
